SEQUENCE LISTING (144 sequences)

SEQ ID NO: 1 - DNA, 699 bp, Nostoc punctiforme PCC 73102
  CDS (1)..(699)
atgcagcagcttacagaccaatctaaagaattagatttcaagagcgaaacatacaaagatgcttatagccggattaatgcgatcgtgattgaaggggaacaagaagcccatgaaaattacatcacactagcccaactgctgccagaatctcatgatgaattgattcgcctatccaagatggaaagccgccataagaaaggatttgaagcttgtgggcgcaatttagctgttaccccagatttgcaatttgccaaagagtttttctccggcctacaccaaaattttcaaacagctgccgcagaagggaaagtggttacttgtctgttgattcagtctttaattattgaatgttttgcgatcgcagcatataacatttacatccccgttgccgacgatttcgcccgtaaaattactgaaggagtagttaaagaagaatacagccacctcaattttggagaagtttggttgaaagaacactttgcagaatccaaagctgaacttgaacttgcaaatcgccagaacctacccatcgtctggaaaatgctcaaccaagtagaaggtgatgcccacacaatggcaatggaaaaagatgctttggtagaagacttcatgattcagtatggtgaagcattgagtaacattggtttttcgactcgcgatattatgcgcttgtcagcctacggactcataggtgcttaa

SEQ ID NO: 2 - protein, 232 aa, Nostoc punctiforme PCC 73102
MQQLTDQSKELDFKSETYKDAYSRINAIVIEGEQEAHENYITLAQLLPESHDELIRLSKMESRHKKGFEACGRNLAVTPDLQFAKEFFSGLHQNFQTAAAEGKVVTCLLIQSLIIECFAIAAYNIYIPVADDFARKITEGVVKEEYSHLNFGEVWLKEHFAESKAELELANRQNLPIVWKMLNQVEGDAHTMAMEKDALVEDFMIQYGEALSNIGFSTRDIMRLSAYGLIGA

SEQ ID NO: 3 - DNA, 1026 bp, Synechococcus elongatus PCC7942
  CDS (1)..(1026)
atgttcggtcttatcggtcatctcaccagtttggagcaggcccgcgacgtttctcgcaggatgggctacgacgaatacgccgatcaaggattggagttttggagtagcgctcctcctcaaatcgttgatgaaatcacagtcaccagtgccacaggcaaggtgattcacggtcgctacatcgaatcgtgtttcttgccggaaatgctggcggcgcgccgcttcaaaacagccacgcgcaaagttctcaatgccatgtcccatgcccaaaaacacggcatcgacatctcggccttggggggctttacctcgattattttcgagaatttcgatttggccagtttgcggcaagtgcgcgacactaccttggagtttgaacggttcaccaccggcaatactcacacggcctacgtaatctgtagacaggtggaagccgctgctaaaacgctgggcatcgacattacccaagcgacagtagcggttgtcggcgcgactggcgatatcggtagcgctgtctgccgctggctcgacctcaaactgggtgtcggtgatttgatcctgacggcgcgcaatcaggagcgtttggataacctgcaggctgaactcggccggggcaagattctgcccttggaagccgctctgccggaagctgactttatcgtgtgggtcgccagtatgcctcagggcgtagtgatcgacccagcaaccctgaagcaaccctgcgtcctaatcgacgggggctaccccaaaaacttgggcagcaaagtccaaggtgagggcatctatgtcctcaatggcggggtagttgaacattgcttcgacatcgactggcagatcatgtccgctgcagagatggcgcggcccgagcgccagatgtttgcctgctttgccgaggcgatgctcttggaatttgaaggctggcatactaacttctcctggggccgcaaccaaatcacgatcgagaagatggaagcgatcggtgaggcatcggtgcgccacggcttccaacccttggcattggcaatttga

SEQ ID NO: 4 - protein, 341 aa, Synechococcus elongatus PCC7942
MFGLIGHLTSLEQARDVSRRMGYDEYADQGLEFWSSAPPQIVDEITVTSATGKVIHGRYIESCFLPEMLAARRFKTATRKVLNAMSHAQKHGIDISALGGFTSIIFENFDLASLRQVRDTTLEFERFTTGNTHTAYVICRQVEAAAKTLGIDITQATVAVVGATGDIGSAVCRWLDLKLGVGDLILTARNQERLDNLQAELGRGKILPLEAALPEADFIVWVASMPQGVVIDPATLKQPCVLIDGGYPKNLGSKVQGEGIYVLNGGVVEHCFDIDWQIMSAAEMARPERQMFACFAEAMLLEFEGWHTNFSWGRNQITIEKMEAIGEASVRHGFQPLALAI

SEQ ID NO: 5 - DNA, 40 bp, Artificial
  Synthetic DNA
cgagctcggcgcgcctgcagatgcagcagcttacagacca

SEQ ID NO: 6 - DNA, 40 bp, Artificial
  Synthetic DNA
gcaagcttgtcgacctgcagttaagcacctatgagtccgt

SEQ ID NO: 7 - DNA, 37 bp, Artificial
  Synthetic DNA
aaggagatatacatatgatgttcggtcttatcggtca

SEQ ID NO: 8 - DNA, 37 bp, Artificial
  Synthetic DNA
ttgagatctgccatatgtcaaattgccaatgccaagg

SEQ ID NO: 9 - DNA, 31 bp, Artificial
  Synthetic DNA
aggagatataccatgcagcagcttacagacc

SEQ ID NO: 10 - DNA, 74 bp, Artificial
  Synthetic DNA
gctcgaattcggatcttacaccacatcatcttcggcacctggcatggcaacgccagcacctatgagtccgtagg

SEQ ID NO: 11 - DNA, 37 bp, Artificial; identical to SEQ ID NO: 7
  Synthetic DNA

SEQ ID NO: 12 - DNA, 37 bp, Artificial; identical to SEQ ID NO: 8
  Synthetic DNA

SEQ ID NO: 13 - DNA, 35 bp, Artificial
  Synthetic DNA
gaaacatttaaagatgcttatagccggattaatgc

SEQ ID NO: 14 - DNA, 38 bp, Artificial
  Synthetic DNA
atctttaaatgtttcgctcttgaaatctaattctttag

SEQ ID NO: 15 - DNA, 32 bp, Artificial
  Synthetic DNA
gcgatcaccattgaaggggaacaagaagccca

SEQ ID NO: 16 - DNA, 33 bp, Artificial
  Synthetic DNA
ttcaatggtgatcgcattaatccggctataagc

SEQ ID NO: 17 - DNA, 32 bp, Artificial
  Synthetic DNA
gcgatcatgattgaaggggaacaagaagccca

SEQ ID NO: 18 - DNA, 33 bp, Artificial
  Synthetic DNA
ttcaatcatgatcgcattaatccggctataagc

SEQ ID NO: 19 - DNA, 34 bp, Artificial
  Synthetic DNA
gaacaatatgcccatgaaaattacatcacactag

SEQ ID NO: 20 - DNA, 31 bp, Artificial
  Synthetic DNA
atgggcatattgttccccttcaatcacgatc

SEQ ID NO: 21 - DNA, 31 bp, Artificial
  Synthetic DNA
catgaaatgtacatcacactagcccaactgc

SEQ ID NO: 22 - DNA, 33 bp, Artificial
  Synthetic DNA
gatgtacatttcatgggcttcttgttccccttc

SEQ ID NO: 23 - DNA, 31 bp, Artificial
  Synthetic DNA
catgaaacctacatcacactagcccaactgc

SEQ ID NO: 24 - DNA, 33 bp, Artificial
  Synthetic DNA
gatgtaggtttcatgggcttcttgttccccttc

SEQ ID NO: 25 - DNA, 31 bp, Artificial
  Synthetic DNA
catgaagtgtacatcacactagcccaactgc

SEQ ID NO: 26 - DNA, 33 bp, Artificial
  Synthetic DNA
gatgtacacttcatgggcttcttgttccccttc

SEQ ID NO: 27 - DNA, 32 bp, Artificial
  Synthetic DNA
tacatcgatctagcccaactgctgccagaatc

SEQ ID NO: 28 - DNA, 36 bp, Artificial
  Synthetic DNA
ggctagatcgatgtaattttcatgggcttcttgttc

SEQ ID NO: 29 - DNA, 32 bp, Artificial
  Synthetic DNA
tacatcaacctagcccaactgctgccagaatc

SEQ ID NO: 30 - DNA, 36 bp, Artificial
  Synthetic DNA
ggctaggttgatgtaattttcatgggcttcttgttc

SEQ ID NO: 31 - DNA, 31 bp, Artificial
  Synthetic DNA
acactaagccaactgctgccagaatctcatg

SEQ ID NO: 32 - DNA, 36 bp, Artificial
  Synthetic DNA
cagttggcttagtgtgatgtaattttcatgggcttc

SEQ ID NO: 33 - DNA, 33 bp, Artificial
  Synthetic DNA
gaatcttatgatgaattgattcgcctatccaag

SEQ ID NO: 34 - DNA, 32 bp, Artificial
  Synthetic DNA
ttcatcataagattctggcagcagttgggcta

SEQ ID NO: 35 - DNA, 33 bp, Artificial
  Synthetic DNA
gatgaacagattcgcctatccaagatggaaagc

SEQ ID NO: 36 - DNA, 33 bp, Artificial
  Synthetic DNA
gcgaatctgttcatcatgagattctggcagcag

SEQ ID NO: 37 - DNA, 33 bp, Artificial
  Synthetic DNA
gatgaagaaattcgcctatccaagatggaaagc

SEQ ID NO: 38 - DNA, 33 bp, Artificial
  Synthetic DNA
gcgaatttcttcatcatgagattctggcagcag

SEQ ID NO: 39 - DNA, 33 bp, Artificial
  Synthetic DNA
gatgaaaaaattcgcctatccaagatggaaagc

SEQ ID NO: 40 - DNA, 33 bp, Artificial
  Synthetic DNA
gcgaattttttcatcatgagattctggcagcag

SEQ ID NO: 41 - DNA, 31 bp, Artificial
  Synthetic DNA
gaattgtggcgcctatccaagatggaaagcc

SEQ ID NO: 42 - DNA, 33 bp, Artificial
  Synthetic DNA
taggcgccacaattcatcatgagattctggcag

SEQ ID NO: 43 - DNA, 33 bp, Artificial
  Synthetic DNA
attcgccagtccaagatggaaagccgccataag

SEQ ID NO: 44 - DNA, 35 bp, Artificial
  Synthetic DNA
cttggactggcgaatcaattcatcatgagattctc

SEQ ID NO: 45 - DNA, 33 bp, Artificial
  Synthetic DNA
attcgcgaatccaagatggaaagccgccataag

SEQ ID NO: 46 - DNA, 35 bp, Artificial
  Synthetic DNA
cttggattcgcgaatcaattcatcatgagattctc

SEQ ID NO: 47 - DNA, 33 bp, Artificial
  Synthetic DNA
attcgcaaatccaagatggaaagccgccataag

SEQ ID NO: 48 - DNA, 35 bp, Artificial
  Synthetic DNA
cttggatttgcgaatcaattcatcatgagattctc

SEQ ID NO: 49 - DNA, 33 bp, Artificial
  Synthetic DNA
attcgctggtccaagatggaaagccgccataag

SEQ ID NO: 50 - DNA, 35 bp, Artificial
  Synthetic DNA
cttggaccagcgaatcaattcatcatgagattctc

SEQ ID NO: 51 - DNA, 33 bp, Artificial
  Synthetic DNA
attcgctattccaagatggaaagccgccataag

SEQ ID NO: 52 - DNA, 35 bp, Artificial
  Synthetic DNA
cttggaatagcgaatcaattcatcatgagattctc

SEQ ID NO: 53 - DNA, 30 bp, Artificial
  Synthetic DNA
cgcctaaacaagatggaaagccgccataag

SEQ ID NO: 54 - DNA, 32 bp, Artificial
  Synthetic DNA
catcttgtttaggcgaatcaattcatcatgag

SEQ ID NO: 55 - DNA, 34 bp, Artificial
  Synthetic DNA
tccaaggatgaaagccgccataagaaaggatttg

SEQ ID NO: 56 - DNA, 33 bp, Artificial
  Synthetic DNA
gctttcatccttggataggcgaatcaattcatc

SEQ ID NO: 57 - DNA, 34 bp, Artificial
  Synthetic DNA
tccaagaacgaaagccgccataagaaaggatttg

SEQ ID NO: 58 - DNA, 33 bp, Artificial
  Synthetic DNA
gctttcgttcttggataggcgaatcaattcatc

SEQ ID NO: 59 - DNA, 31 bp, Artificial
  Synthetic DNA
aagatgaccagccgccataagaaaggatttg

SEQ ID NO: 60 - DNA, 33 bp, Artificial
  Synthetic DNA
gcggctggtcatcttggataggcgaatcaattc

SEQ ID NO: 61 - DNA, 32 bp, Artificial
  Synthetic DNA
cgccattataaaggatttgaagcttgtgggcg

SEQ ID NO: 62 - DNA, 33 bp, Artificial
  Synthetic DNA
tcctttataatggcggctttccatcttggatag

SEQ ID NO: 63 - DNA, 34 bp, Artificial
  Synthetic DNA
aagaaacattttgaagcttgtgggcgcaatttag

SEQ ID NO: 64 - DNA, 31 bp, Artificial
  Synthetic DNA
ttcaaaatgtttcttatggcggctttccatc

SEQ ID NO: 65 - DNA, 32 bp, Artificial
  Synthetic DNA
tttgaaagctgtgggcgcaatttagctgttac

SEQ ID NO: 66 - DNA, 32 bp, Artificial
  Synthetic DNA
cccacagctttcaaatcctttcttatggcggc

SEQ ID NO: 67 - DNA, 30 bp, Artificial
  Synthetic DNA
gaagctggcgggcgcaatttagctgttacc

SEQ ID NO: 68 - DNA, 32 bp, Artificial
  Synthetic DNA
gcgcccgccagcttcaaatcctttcttatggc

SEQ ID NO: 69 - DNA, 34 bp, Artificial
  Synthetic DNA
tttttcaacggcctacaccaaaattttcaaacag

SEQ ID NO: 70 - DNA, 36 bp, Artificial
  Synthetic DNA
taggccgttgaaaaactctttggcaaattgcaaatc

SEQ ID NO: 71 - DNA, 31 bp, Artificial
  Synthetic DNA
caccaagtgtttcaaacagctgccgcagaag

SEQ ID NO: 72 - DNA, 33 bp, Artificial
  Synthetic DNA
ttgaaacacttggtgtaggccggagaaaaactc

SEQ ID NO: 73 - DNA, 31 bp, Artificial
  Synthetic DNA
gctgccagcgaagggaaagtggttacttgtc

SEQ ID NO: 74 - DNA, 33 bp, Artificial
  Synthetic DNA
cccttcgctggcagctgtttgaaaattttggtg

SEQ ID NO: 75 - DNA, 32 bp, Artificial
  Synthetic DNA
gggaaagcggttacttgtctgttgattcagtc

SEQ ID NO: 76 - DNA, 33 bp, Artificial
  Synthetic DNA
agtaaccgctttcccttctgcggcagctgtttg

SEQ ID NO: 77 - DNA, 32 bp, Artificial
  Synthetic DNA
gggaaaaacgttacttgtctgttgattcagtc

SEQ ID NO: 78 - DNA, 33 bp, Artificial
  Synthetic DNA
agtaacgtttttcccttctgcggcagctgtttg

SEQ ID NO: 79 - DNA, 37 bp, Artificial
  Synthetic DNA
gtggttgtgtgtctgttgattcagtctttaattattg

SEQ ID NO: 80 - DNA, 29 bp, Artificial
  Synthetic DNA
cagacacacaaccactttcccttctgcgg

SEQ ID NO: 81 - DNA, 37 bp, Artificial
  Synthetic DNA
gtggttatgtgtctgttgattcagtctttaattattg

SEQ ID NO: 82 - DNA, 29 bp, Artificial
  Synthetic DNA
cagacacataaccactttcccttctgcgg

SEQ ID NO: 83 - DNA, 36 bp, Artificial
  Synthetic DNA
ttgattctgtctttaattattgaatgttttgcgatc

SEQ ID NO: 84 - DNA, 35 bp, Artificial
  Synthetic DNA
taaagacagaatcaacagacaagtaaccactttcc

SEQ ID NO: 85 - DNA, 36 bp, Artificial
  Synthetic DNA
ttgattatttctttaattattgaatgttttgcgatc

SEQ ID NO: 86 - DNA, 35 bp, Artificial
  Synthetic DNA
taaagaaataatcaacagacaagtaaccactttcc

SEQ ID NO: 87 - DNA, 34 bp, Artificial
  Synthetic DNA
gcagcacataacatttacatccccgttgccgacg

SEQ ID NO: 88 - DNA, 30 bp, Artificial
  Synthetic DNA
aatgttatgtgctgcgatcgcaaaacattc

SEQ ID NO: 89 - DNA, 31 bp, Artificial
  Synthetic DNA
aacattcatatccccgttgccgacgatttcg

SEQ ID NO: 90 - DNA, 35 bp, Artificial
  Synthetic DNA
ggggatatgaatgttatatgctgcgatcgcaaaac

SEQ ID NO: 91 - DNA, 31 bp, Artificial
  Synthetic DNA
gttaaagatgaatacagccacctcaattttg

SEQ ID NO: 92 - DNA, 35 bp, Artificial
  Synthetic DNA
gtattcatctttaactactccttcagtaattttac

SEQ ID NO: 93 - DNA, 31 bp, Artificial
  Synthetic DNA
gttaaagtggaatacagccacctcaattttg

SEQ ID NO: 94 - DNA, 35 bp, Artificial
  Synthetic DNA
gtattccactttaactactccttcagtaattttac

SEQ ID NO: 95 - DNA, 31 bp, Artificial
  Synthetic DNA
ggagaagaatggttgaaagaacactttgcag

SEQ ID NO: 96 - DNA, 31 bp, Artificial
  Synthetic DNA
caaccattcttctccaaaattgaggtggctg

SEQ ID NO: 97 - DNA, 31 bp, Artificial
  Synthetic DNA
cactttagcgaatccaaagctgaacttgaac

SEQ ID NO: 98 - DNA, 33 bp, Artificial
  Synthetic DNA
ggattcgctaaagtgttctttcaaccaaacttc

SEQ ID NO: 99 - DNA, 33 bp, Artificial
  Synthetic DNA
cttgaaaccgcaaatcgccagaacctacccatc

SEQ ID NO: 100 - DNA, 39 bp, Artificial
  Synthetic DNA
atttgcggtttcaagttcagctttggattctgcaaagtg

SEQ ID NO: 101 - DNA, 33 bp, Artificial
  Synthetic DNA
cttgaagcggcaaatcgccagaacctacccatc

SEQ ID NO: 102 - DNA, 32 bp, Artificial
  Synthetic DNA
atttgccgcttcaagttcagctttggattctg

SEQ ID NO: 103 - DNA, 33 bp, Artificial
  Synthetic DNA
cttgaatatgcaaatcgccagaacctacccatc

SEQ ID NO: 104 - DNA, 39 bp, Artificial
  Synthetic DNA
atttgcatattcaagttcagctttggattctgcaaagtg

SEQ ID NO: 105 - DNA, 31 bp, Artificial
  Synthetic DNA
gaattgtatcgcctatccaagatggaaagcc

SEQ ID NO: 106 - DNA, 33 bp, Artificial
  Synthetic DNA
taggcgatacaattcatcatgagattctggcag

SEQ ID NO: 107 - DNA, 30 bp, Artificial
  Synthetic DNA
gcatatgtgatttacatccccgttgccgac

SEQ ID NO: 108 - DNA, 33 bp, Artificial
  Synthetic DNA
gtaaatcacatatgctgcgatcgcaaaacattc

SEQ ID NO: 109 - DNA, 33 bp, Artificial
  Synthetic DNA
cttgaatgggcaaatcgccagaacctacccatc

SEQ ID NO: 110 - DNA, 37 bp, Artificial
  Synthetic DNA
atttgcccattcaagttcagctttggattctgcaaag

SEQ ID NO: 111 - DNA, 31 bp, Artificial
  Synthetic DNA
cgccagatgctacccatcgtctggaaaatgc

SEQ ID NO: 112 - DNA, 33 bp, Artificial
  Synthetic DNA
gggtagcatctggcgatttgcaagttcaagttc

SEQ ID NO: 113 - DNA, 31 bp, Artificial
  Synthetic DNA
cgccagaccctacccatcgtctggaaaatgc

SEQ ID NO: 114 - DNA, 30 bp, Artificial
  Synthetic DNA
gggtagggtctggcgatttgcaagttcaag

SEQ ID NO: 115 - DNA, 33 bp, Artificial
  Synthetic DNA
cagaaccagcccatcgtctggaaaatgctcaac

SEQ ID NO: 116 - DNA, 33 bp, Artificial
  Synthetic DNA
gatgggctggttctggcgatttgcaagttcaag

SEQ ID NO: 117 - DNA, 33 bp, Artificial
  Synthetic DNA
cagaacgaacccatcgtctggaaaatgctcaac

SEQ ID NO: 118 - DNA, 33 bp, Artificial
  Synthetic DNA
gatgggttcgttctggcgatttgcaagttcaag

SEQ ID NO: 119 - DNA, 37 bp, Artificial
  Synthetic DNA
cagaacaaacccatcgtctggaaaatgctcaaccaag

SEQ ID NO: 120 - DNA, 33 bp, Artificial
  Synthetic DNA
gatgggtttgttctggcgatttgcaagttcaag

SEQ ID NO: 121 - DNA, 34 bp, Artificial
  Synthetic DNA
ctaccctatgtctggaaaatgctcaaccaagtag

SEQ ID NO: 122 - DNA, 33 bp, Artificial
  Synthetic DNA
ccagacatagggtaggttctggcgatttgcaag

SEQ ID NO: 123 - DNA, 34 bp, Artificial
  Synthetic DNA
ctaccctgggtctggaaaatgctcaaccaagtag

SEQ ID NO: 124 - DNA, 39 bp, Artificial
  Synthetic DNA
ccagacccagggtaggttctggcgatttgcaagttcaag

SEQ ID NO: 125 - DNA, 32 bp, Artificial
  Synthetic DNA
gaaggtgtggcccacacaatggcaatggaaaa

SEQ ID NO: 126 - DNA, 35 bp, Artificial
  Synthetic DNA
gtgggccacaccttctacttggttgagcattttcc

SEQ ID NO: 127 - DNA, 34 bp, Artificial
  Synthetic DNA
gcccacgtgatggcaatggaaaaagatgctttgg

SEQ ID NO: 128 - DNA, 37 bp, Artificial
  Synthetic DNA
tgccatcacgtgggcatcaccttctacttggttgagc

SEQ ID NO: 129 - DNA, 34 bp, Artificial
  Synthetic DNA
gcccacattatggcaatggaaaaagatgctttgg

SEQ ID NO: 130 - DNA, 31 bp, Artificial
  Synthetic DNA
tgccataatgtgggcatcaccttctacttgg

SEQ ID NO: 131 - DNA, 32 bp, Artificial
  Synthetic DNA
cacacactggcaatggaaaaagatgctttggt

SEQ ID NO: 132 - DNA, 33 bp, Artificial
  Synthetic DNA
cattgccagtgtgtgggcatcaccttctacttg

SEQ ID NO: 133 - DNA, 37 bp, Artificial
  Synthetic DNA
atggaactggatgctttggtagaagacttcatgattc

SEQ ID NO: 134 - DNA, 30 bp, Artificial
  Synthetic DNA
agcatccagttccattgccattgtgtgggc

SEQ ID NO: 135 - DNA, 37 bp, Artificial
  Synthetic DNA
atggaaattgatgctttggtagaagacttcatgattc

SEQ ID NO: 136 - DNA, 33 bp, Artificial
  Synthetic DNA
agcatcaatttccattgccattgtgtgggcatc

SEQ ID NO: 137 - DNA, 30 bp, Artificial
  Synthetic DNA
gaaaaagaagctttggtagaagacttcatg

SEQ ID NO: 138 - DNA, 36 bp, Artificial
  Synthetic DNA
caaagcttctttttccattgccattgtgtgggcatc

SEQ ID NO: 139 - DNA, 32 bp, Artificial
  Synthetic DNA
gaaaaatatgctttggtagaagacttcatgat

SEQ ID NO: 140 - DNA, 31 bp, Artificial
  Synthetic DNA
caaagcatatttttccattgccattgtgtgg

SEQ ID NO: 141 - DNA, 31 bp, Artificial
  Synthetic DNA
ttggtatatgacttcatgattcagtatggtg

SEQ ID NO: 142 - DNA, 32 bp, Artificial
  Synthetic DNA
gaagtcatataccaaagcatctttttccattg

SEQ ID NO: 143 - DNA, 29 bp, Artificial
  Synthetic DNA
cagtatcatgaagcattgagtaacattgg

SEQ ID NO: 144 - DNA, 34 bp, Artificial
  Synthetic DNA
tgcttcatgatactgaatcatgaagtcttctacc

(19) United States
(12) Patent Application Publication
(10) Pub. No.: US 2020/0165619 A1
(43) Pub. Date: May 28, 2020

(54) MUTANT DECARBONYLASE GENE, RECOMBINANT MICROORGANISM HAVING THE MUTANT DECARBONYLASE GENE, AND METHOD FOR PRODUCING ALKANE

(71) Applicant: TOYOTA JIDOSHA KABUSHIKI KAISHA, Toyota-shi (JP)

(72) Inventors: Masayoshi Muramatsu, Miyoshi-shi (JP); Shusei Obata, Nagoya-shi (JP)

(73) Assignee: TOYOTA JIDOSHA KABUSHIKI KAISHA, Toyota-shi (JP)

(21) Appl. No.: 16/689,551

(22) Filed: Nov. 20, 2019

(30) Foreign Application Priority Data
Nov. 22, 2018 (JP) 2018-218879

(51) Int. Cl. C12N 15/63; C12P 5/02

(52) U.S. Cl. CPC C12N 15/63; C12P 5/026

(57) ABSTRACT

The present disclosure is intended to identify a substitution mutation that improves enzyme activity of a decarbonylase. Such substitution mutation is implemented at valine at position 29, glutamic acid at position 35, asparagine at position 39, threonine at position 42, histidine at position 51, leucine at position 54, methionine at position 60, serine at position 89, asparagine at position 94, leucine at position 169, asparagine at position 174, leucine at position 175, isoleucine at position 177, or aspartic acid at position 188 in the amino acid sequence as shown in SEQ ID NO: 2.

### CROSS REFERENCE TO RELATED APPLICATIONS

The present application claims priority from Japanese patent application JP 2018-218879 filed on Nov. 22, 2018, the content of which is hereby incorporated by reference into this application.

### BACKGROUND

### Technical Field

The present disclosure relates to a mutant decarbonylase gene encoding a decarbonylase mutant having a substitution mutation of an amino acid, a recombinant microorganism having such mutant decarbonylase gene, and a method for producing alkane.

### Background Art

Alkane is contained in petroleum, it is purified by fractional distillation, and it is used for a wide variety of applications. In addition, alkane is extensively used as a raw material in chemical industry, and it is also a main component of a diesel fuel obtained from petroleum. In recent years, a technique of coexpressing an acyl ACP reductase gene derived from blue-green algae and a decarbonylase gene in E. coli and producing alkane, which is a light oil component, via fermentation has been developed (U.S. Pat. No. 8,846,371).

A decarbonylase, which is a key enzyme in alkane synthesis, is reported to need ferredoxin and ferredoxin reductase to exert its activity (Science, Vol. 329, pp. 559-562, 2010; and WO 2013/024527). When synthesizing alkane with Saccharomyces cerevisae, it is reported that the E. coli-derived ferredoxin gene and the ferredoxin reductase gene are required to be expressed in addition to the decarbonylase gene (Biotechnology Bioengineering, Vol. 112, No. 6, pp. 1275-1279, 2015). According to Biotechnology Bioengineering, Vol. 112, No. 6, pp. 1275-1279, 2015, the amount of alkane produced is approximately 3 μg/g dry cells. In this case, Saccharomyces cerevisae has an O.D. 600 nm of approximately 20 at full growth, and the dry cell weight is approximately 4 g of dry cells/l. On the basis thereof, the amount of production is understood to be as low as approximately 12 μg/l according to the method disclosed in Biotechnology Bioengineering, Vol. 112, No. 6, pp. 1275-1279, 2015.

It has been pointed out that activity of decarbonylase is lowered or lost by hydrogen peroxide produced at the time of the reaction (Proceedings of the National Academy of Sciences of the United States of America, 110, 8, 2013, 3191-3196). According to Proceedings of the National Academy of Sciences of the United States of America, 110, 8, 2013, 3191-3196, the activity lowered or lost because of hydrogen peroxide can be improved in the form of a fusion protein of a decarbonylase and a catalase. Also, a decarbonylase has been subjected to analysis in terms of crystalline structure, and information concerning the enzyme reaction mechanism and the amino acid residues involved in reactions has been elucidated (Biochemical and Biophysical Research Communications, 477, 2016, 395-400; and Protein Cell 6, 1, 2015, 55-67).

### SUMMARY

A conventional decarbonylase was insufficient in terms of enzyme activity. Under the above circumstances, accordingly, the present disclosure is intended to identify substitution mutations that improve enzyme activity of a decarbonylase and to provide a mutant decarbonylase gene encoding a decarbonylase having such mutation substitution(s), a recombinant microorganism having such mutant decarbonylase gene, and a method for producing alkane.

We have conducted concentrated studies in order to overcome the problems indicated above. As a result, we discovered that enzyme activity could be improved to a significant extent by substitution of a particular amino acid residue(s) of a decarbonylase, thereby leading to the completion of the present disclosure.

Specifically, the present disclosure includes the following.

(1) A mutant decarbonylase gene encoding a decarbonylase having at least one substitution mutation, wherein the substitution mutation is selected from the group consisting of:

a substitution mutation of an amino acid corresponding to valine at position 29 in the amino acid sequence as shown in SEQ ID NO: 2 with an amino acid with a low degree of hydrophobicity;

a substitution mutation of an amino acid corresponding to glutamic acid at position 35 in the amino acid sequence as shown in SEQ ID NO: 2 with an amino acid with a high degree of hydrophobicity;

a substitution mutation of an amino acid corresponding to asparagine at position 39 in the amino acid sequence as shown in SEQ ID NO: 2 with an amino acid with a high degree of hydrophobicity;

a substitution mutation of an amino acid corresponding to threonine at position 42 in the amino acid sequence as shown in SEQ ID NO: 2 with an amino acid with a low degree of hydrophobicity;

a substitution mutation of an amino acid corresponding to histidine at position 51 in the amino acid sequence as shown in SEQ ID NO: 2 with an amino acid with a high degree of hydrophobicity;

a substitution mutation of an amino acid corresponding to leucine at position 54 in the amino acid sequence as shown in SEQ ID NO: 2 with an amino acid with a low degree of hydrophobicity;

a substitution mutation of an amino acid corresponding to methionine at position 60 in the amino acid sequence as shown in SEQ ID NO: 2 with an amino acid with a low degree of hydrophobicity;

a substitution mutation of an amino acid corresponding to serine at position 89 in the amino acid sequence as shown in SEQ ID NO: 2 with an amino acid with a low degree of hydrophobicity;

a substitution mutation of an amino acid corresponding to asparagine at position 94 in the amino acid sequence as shown in SEQ ID NO: 2 with an amino acid with a high degree of hydrophobicity;

a substitution mutation of an amino acid corresponding to leucine at position 169 in the amino acid sequence as shown in SEQ ID NO: 2 with an amino acid with a low degree of hydrophobicity;

a substitution mutation of an amino acid corresponding to asparagine at position 174 in the amino acid sequence as shown in SEQ ID NO: 2 with an amino acid with a high degree of hydrophobicity;

a substitution mutation of an amino acid corresponding to leucine at position 175 in the amino acid sequence as shown in SEQ ID NO: 2 with an amino acid with a low degree of hydrophobicity;

a substitution mutation of an amino acid corresponding to isoleucine at position 177 in the amino acid sequence as shown in SEQ ID NO: 2 with an amino acid with a low degree of hydrophobicity; and

a substitution mutation of an amino acid corresponding to aspartic acid at position 188 in the amino acid sequence as shown in SEQ ID NO: 2 with an amino acid with a high degree of hydrophobicity.

(2) The mutant decarbonylase gene according to (1), wherein the substitution mutation of an amino acid corresponding to valine at position 29 is a substitution mutation with an amino acid selected from the group consisting of Y, W, S, G, A, M, C, F, and L.

(3) The mutant decarbonylase gene according to (1), wherein the substitution mutation of an amino acid corresponding to valine at position 29 is a substitution mutation with an amino acid selected from the group consisting of Y, W, S, G, A, and M.

(4) The mutant decarbonylase gene according to (1), wherein the substitution mutation of an amino acid corresponding to valine at position 29 is a substitution mutation with M.

(5) The mutant decarbonylase gene according to (1), wherein the substitution mutation of an amino acid corresponding to glutamic acid at position 35 is a substitution mutation with an amino acid selected from the group consisting of P, Y, W, S, T, G, A, M, C, F, L, V, and I.

(6) The mutant decarbonylase gene according to (1), wherein the substitution mutation of an amino acid corresponding to glutamic acid at position 35 is a substitution mutation with an amino acid selected from the group consisting of P, Y, W, S, T, and G.

(7) The mutant decarbonylase gene according to (1), wherein the substitution mutation of an amino acid corresponding to glutamic acid at position 35 is a substitution mutation with Y.

(8) The mutant decarbonylase gene according to (1), wherein the substitution mutation of an amino acid corresponding to asparagine at position 39 is a substitution mutation with an amino acid selected from the group consisting of G, A, C, F, L, V, and I.

(9) The mutant decarbonylase gene according to (1), wherein the substitution mutation of an amino acid corresponding to asparagine at position 39 is a substitution mutation with an amino acid selected from the group consisting of C, F, L, V, and I.

(10) The mutant decarbonylase gene according to (1), wherein the substitution mutation of an amino acid corresponding to asparagine at position 39 is a substitution mutation with V.

(11) The mutant decarbonylase gene according to (1), wherein the substitution mutation of an amino acid corresponding to threonine at position 42 is a substitution mutation with an amino acid selected from the group consisting of R, K, Q, N, D, E, H, P, Y, W, S, and G.

(12) The mutant decarbonylase gene according to (1), wherein the substitution mutation of an amino acid corresponding to threonine at position 42 is a substitution mutation with an amino acid selected from the group consisting of K, Q, N, D, E, H, P, and Y.

(13) The mutant decarbonylase gene according to (1), wherein the substitution mutation of an amino acid corresponding to threonine at position 42 is a substitution mutation with N or D.

(14) The mutant decarbonylase gene according to (1), wherein the substitution mutation of an amino acid corresponding to histidine at position 51 is a substitution mutation with an amino acid selected from the group consisting of P, Y, W, S, T, and G.

(15) The mutant decarbonylase gene according to (1), wherein the substitution mutation of an amino acid corresponding to histidine at position 51 is a substitution mutation with P or Y.

(16) The mutant decarbonylase gene according to (1), wherein the substitution mutation of an amino acid corresponding to histidine at position 51 is a substitution mutation with Y.

(17) The mutant decarbonylase gene according to (1), wherein the substitution mutation of an amino acid corresponding to leucine at position 54 is a substitution mutation with an amino acid selected from the group consisting of Q, N, D, H, P, Y, W, S, T, and G.

(18) The mutant decarbonylase gene according to (1), wherein the substitution mutation of an amino acid corresponding to leucine at position 54 is a substitution mutation with an amino acid selected from the group consisting of Q, N, D, H, P, and Y.

(19) The mutant decarbonylase gene according to (1), wherein the substitution mutation of an amino acid corresponding to leucine at position 54 is a substitution mutation with Q.

(20) The mutant decarbonylase gene according to (1), wherein the substitution mutation of an amino acid corresponding to methionine at position 60 is a substitution mutation with an amino acid selected from the group consisting of Q, D, E, H, P, and Y.

(21) The mutant decarbonylase gene according to (1), wherein the substitution mutation of an amino acid corresponding to methionine at position 60 is a substitution mutation with an amino acid selected from the group consisting of Q, D, and E.

(22) The mutant decarbonylase gene according to (1), wherein the substitution mutation of an amino acid corresponding to methionine at position 60 is a substitution mutation with D.

(23) The mutant decarbonylase gene according to (1), wherein the substitution mutation of an amino acid corresponding to serine at position 89 is a substitution mutation with an amino acid selected from the group consisting of Q, N, D, E, H, P, and Y.

(24) The mutant decarbonylase gene according to (1), wherein the substitution mutation of an amino acid corresponding to serine at position 89 is a substitution mutation with an amino acid selected from the group consisting of Q, N, D, and E.

(25) The mutant decarbonylase gene according to (1), wherein the substitution mutation of an amino acid corresponding to serine at position 89 is a substitution mutation with N.

(26) The mutant decarbonylase gene according to (1), wherein the substitution mutation of an amino acid corresponding to asparagine at position 94 is a substitution mutation with an amino acid selected from the group consisting of C, F, L, V, and I.

(27) The mutant decarbonylase gene according to (1), wherein the substitution mutation of an amino acid corresponding to asparagine at position 94 is a substitution mutation with an amino acid selected from the group consisting of L, V, and I.

(28) The mutant decarbonylase gene according to (1), wherein the substitution mutation of an amino acid corresponding to asparagine at position 94 is a substitution mutation with V.

(29) The mutant decarbonylase gene according to (1), wherein the substitution mutation of an amino acid corresponding to leucine at position 169 is a substitution mutation with an amino acid selected from the group consisting of P, Y, W, S, T, G, A, and M.

(30) The mutant decarbonylase gene according to (1), wherein the substitution mutation of an amino acid corresponding to leucine at position 169 is a substitution mutation with an amino acid selected from the group consisting of Y, W, S, T, G, and A.

(31) The mutant decarbonylase gene according to (1), wherein the substitution mutation of an amino acid corresponding to leucine at position 169 is a substitution mutation with an amino acid selected from the group consisting of Y, W, and A.

(32) The mutant decarbonylase gene according to (1), wherein the substitution mutation of an amino acid corresponding to asparagine at position 174 is a substitution mutation with an amino acid selected from the group consisting of P, Y, W, S, T, G, A, M, C, and F.

(33) The mutant decarbonylase gene according to (1), wherein the substitution mutation of an amino acid corresponding to asparagine at position 174 is a substitution mutation with an amino acid selected from the group consisting of W, S, T, G, A, and M.

(34) The mutant decarbonylase gene according to (1), wherein the substitution mutation of an amino acid corresponding to asparagine at position 174 is a substitution mutation with T or M.

(35) The mutant decarbonylase gene according to (1), wherein the substitution mutation of an amino acid corresponding to leucine at position 175 is a substitution mutation with an amino acid selected from the group consisting of R, K, Q, N, D, E, H, P, and Y.

(36) The mutant decarbonylase gene according to (1), wherein the substitution mutation of an amino acid corresponding to leucine at position 175 is a substitution mutation with an amino acid selected from the group consisting of K, Q, N, D, E, and H.

(37) The mutant decarbonylase gene according to (1), wherein the substitution mutation of an amino acid corresponding to leucine 175 at position is a substitution mutation with an amino acid selected from the group consisting of K, Q, and E.

(38) The mutant decarbonylase gene according to (1), wherein the substitution mutation of an amino acid corresponding to isoleucine 177 at position is a substitution mutation with an amino acid selected from the group consisting of Q, N, D, E, H, P, Y, W, S, T, G, A, and M.

(39) The mutant decarbonylase gene according to (1), wherein the substitution mutation of an amino acid corresponding to isoleucine 177 at position is a substitution mutation with an amino acid selected from the group consisting of P, Y, W, S, T, and G.

(40) The mutant decarbonylase gene according to (1), wherein the substitution mutation of an amino acid corresponding to isoleucine 177 at position is a substitution mutation with Y or W.

(41) The mutant decarbonylase gene according to (1), wherein the substitution mutation of an amino acid corresponding to aspartic acid 188 at position is a substitution mutation with an amino acid selected from the group consisting of C, F, L, V, and I.

(42) The mutant decarbonylase gene according to (1), wherein the substitution mutation of an amino acid corresponding to aspartic acid 188 at position is a substitution mutation with an amino acid selected from the group consisting of L, V, and I.

(43) The mutant decarbonylase gene according to (1), wherein the substitution mutation of an amino acid corresponding to aspartic acid 188 at position is a substitution mutation with V.

(44) The mutant decarbonylase gene according to (1), which has at least one substitution mutation selected from the group consisting of V29M, E35Y, N39T, N39V, T42D, T42N, H51Y, L54Q, M60D, S89N, N94V, L169A, L169Y, L169W, N174M, N174T, L175Q, L175E, L175K, I177Y, I177W, and D188V in the amino acid sequence as shown in SEQ ID NO: 2.

(45) The mutant decarbonylase gene according to (1), which has H51Y and/or L169W in the amino acid sequence as shown in SEQ ID NO: 2.

(46) A recombinant microorganism comprising the mutant decarbonylase gene according to any of (1) to (45) introduced into a host microorganism.

(47) The recombinant microorganism according to (46), wherein the host microorganism is Escherichia coli or a bacterium of the genus Klebsiella.

(48) A method for producing alkane comprising culturing the recombinant microorganism according to (46) or (47).

(49) The method for producing alkane according to (48), which further comprises recovering alkane from a medium in which the recombinant microorganism is cultured.

(50) The method for producing alkane according to (48), which further comprises recovering alkane from a medium in which the recombinant microorganism is cultured and purifying the recovered alkane.

(51) The method for producing alkane according to (48), which further comprises producing alkane having 9 to 20 carbon atoms.

The mutant decarbonylase gene according to the present disclosure encodes a protein having decarbonylase activity superior to that of a wild-type decarbonylase without a mutation. With the use of the mutant decarbonylase gene according to the present disclosure, accordingly, a recombinant microorganism excellent in the alkane-synthesizing capacity can be obtained. In addition, alkane productivity in an alkane synthesis system that involves the use of a recombinant microorganism into which the mutant decarbonylase gene according to the present disclosure has been introduced can be improved to a significant extent, and the cost incurred in alkane production can be reduced to a significant extent.

### BRIEF DESCRIPTION OF THE DRAWINGS

FIG. 1 schematically shows 10 α helix structures (Helix 1 to Helix 10, underlined) and amino acid residues to be substituted (indicated by arrows) in the amino acid sequence of the decarbonylase derived from the N. punctiforme PCC 73102 strain (SEQ ID NO: 2).

FIG. 2 shows amino acid sequences constituting an α helix structure arranged in circles so as to observe, in the axial direction, the α helix structure of Helix 1, in the decarbonylase derived from the N. punctiforme PCC 73102 strain.

FIG. 3 shows amino acid sequences constituting an α helix structure arranged in circles so as to observe, in the axial direction, the α helix structure of Helix 2, in the decarbonylase derived from the N. punctiforme PCC 73102 strain.

FIG. 4 shows amino acid sequences constituting an α helix structure arranged in circles so as to observe, in the axial direction, the α helix structure of Helix 3, in the decarbonylase derived from the N. punctiforme PCC 73102 strain.

FIG. 5 shows amino acid sequences constituting an α helix structure arranged in circles so as to observe, in the axial direction, the α helix structure of Helix 8, in the decarbonylase derived from the N. punctiforme PCC 73102 strain.

FIG. 6 shows a table summarizing the degrees of hydrophobicity of amino acids.

FIG. 7 shows a characteristic diagram demonstrating the results of measuring the amount of hydrocarbons (pentadecane and heptadecane) produced by a transformant comprising a substitution mutation introduced into Helix 1.

FIG. 8 shows a characteristic diagram demonstrating the results of measuring the amount of hydrocarbons (pentadecane and heptadecane) produced by a transformant comprising a substitution mutation introduced into Helix 2.

FIG. 9 shows a characteristic diagram demonstrating the results of measuring the amount of hydrocarbons (pentadecane and heptadecane) produced by a transformant comprising a substitution mutation introduced into Helix 3 or 4.

FIG. 10 shows a characteristic diagram demonstrating the results of measuring the amount of hydrocarbons (pentadecane and heptadecane) produced by a transformant comprising a substitution mutation introduced into Helix 5, 6, or 7.

FIG. 11 shows a characteristic diagram demonstrating the results of measuring the amount of hydrocarbons (pentadecane and heptadecane) produced by a transformant comprising a substitution mutation introduced into Helix 8.

FIG. 12 shows a characteristic diagram demonstrating the results of measuring the amount of hydrocarbons (pentadecane and heptadecane) produced by a transformant comprising a substitution mutation introduced into Helix 9.

### DETAILED DESCRIPTION

Hereafter, the present disclosure is described in greater detail with reference to the figures and the examples.

The mutant decarbonylase gene according to the present disclosure (hereafter, simply referred to as “the mutant decarbonylase gene”) encodes a decarbonylase mutant prepared by introducing a given substitution mutation into a wild-type decarbonylase. In particular, the decarbonylase mutant comprising a substitution mutation introduced thereinto exhibits decarbonylase activity superior to that of a decarbonylase before introduction of the mutation (e.g., a wild-type decarbonylase). The term “decarbonylase activity” used herein refers to activity of decarbonylating an aldehyde compound serving as a substrate to produce a hydrocarbon. Thus, decarbonylase activity can be evaluated based on the amount of hydrocarbon produced.

The term “a substitution mutation” used herein refers to a mutation that causes substitution of a given amino acid residue included in an α helix constituting a decarbonylase with another amino acid, and an amino acid residue to be substituted is selected from among amino acid residues that may deteriorate stability of the α helix structure. More specifically, amino acid residues to be substituted are selected from among amino acid residues exhibiting hydrophilic and/or hydrophobic properties different from other amino acid residues located in the vicinity when the α helix structure is formed.

The amino acid residue to be substituted may be substituted with another amino acid for mutation, so that the resulting mutant would exhibit decarbonylase activity superior to that before the mutation. In such a case, the amino acid residues after the mutation can be arbitrarily selected from among amino acid residues exhibiting more similar hydrophilic and/or hydrophobic properties than amino acid residues before the mutation, compared with other amino acid residues located in the vicinity when the α helix structure is formed.

Concerning hydrophilic and/or hydrophobic properties, the hydropathy index (the degree of hydrophobicity; also referred to as “the hydrophobicity scale”) described in, for example, Kyte J. & Doolittle R F, 1982, J. Mol. Biol., 157: 105-132 can be employed. Hydrophilic and/or hydrophobic properties are not limited to the degree of hydrophobicity defined by Kyte J. & Doolittle R F. For example, the degree of hydrophobicity disclosed in Hopp T P, Woods K R, 1983, Mol. Immunol., 20 (4): 483-489 or the degree of hydrophobicity disclosed in Engelman D M, Steitz T A, Goldman A, 1986, Annu. Rev. Biophys. Biophys. Chem., 15: 321-353 can be adequately employed.

Specifically, amino acid residues after the mutation can be selected from among amino acid residues with the degree of hydrophobicity close to that of other amino acid residues located in the vicinity of the amino acid residues to be substituted when the α helix structure is formed. When given amino acid residues exhibit the degrees of hydrophobicity lower than other amino acid residues located in the vicinity thereof when the α helix structure is formed, for example, the amino acid residues are to be substituted with amino acids with the degree of hydrophobicity higher than the amino acid residues before substitution. When the degree of hydrophobicity of the given amino acid residues is not within a given range deviated from the average degree of hydrophobicity of other amino acid residues located in the vicinity thereof when the α helix structure is formed (e.g., within ±0.15 in terms of the degree of hydrophobicity defined by Kyte and Doolittle), amino acid residues after the substitution can be selected from among amino acids exhibiting the degree of hydrophobicity within such range.

Concerning given amino acid residues, other amino acid residues located in the vicinity thereof when the α helix structure is formed can be defined as amino acid residues arranged in the axial direction of the α helix structure. When amino acid sequences constituting the α helix structure are arranged in circles so as to observe the α helix structure in the axial direction, amino acid residues adjacent to each other are positioned in a direction away from each other by approximately 100 degrees. By arranging the amino acid sequences constituting the α helix structure in circles while maintaining such positional relationship, amino acid residues arranged in the axial direction of the α helix structure can be visually recognized. Specifically, amino acid residues positioned to be adjacent to each other in a circle can be regarded as amino acid residues arranged in the axial direction of the α helix structure. When a given amino acid in an amino acid sequence is designated as amino acid at position 1, more specifically, amino acid at position 5, amino acid at position 8, amino acid at position 12, amino acid at position 19, amino acid at position 26, and amino acid at position 30 are positioned to be adjacent to each other in a circle, and such amino acids can be defined as the amino acids located in the vicinity of amino acid at position 1.

Accordingly, other amino acid residues located in the vicinity of a given amino acid residue included in the α helix structure can be amino acid at position 5, amino acid at position 8, amino acid at position 12, amino acid at position 15, amino acid at position 19, amino acid at position 26, and amino acid at position 30 arranged in the N terminal and/or C terminal direction(s), when the given amino acid residue is designated as amino acid 1. When a given amino acid residue is designated as amino acid at position 1, further, other amino acid residues located in the vicinity of the given amino acid residue can be amino acid at position 8, amino acid at position 12, amino acid at position 19, amino acid at position 26, and amino acid at position 30 arranged in the N terminal and/or C terminal direction(s). When a given amino acid is designated as amino acid at position 1, in addition, other amino acid residues located in the vicinity of the given amino acid residue can be amino acid at position 8, amino acid at position 12, and amino acid at position 19 arranged in the N terminal and/or C terminal direction(s).

Hereafter, an amino acid residue to be substituted is described based on the amino acid sequence of a wild-type decarbonylase. For example, SEQ ID NO: 2 shows the amino acid sequence of the wild-type decarbonylase encoded by the decarbonylase gene derived from the N. punctiforme PCC 73102 strain. SEQ ID NO: 1 shows the nucleotide sequence of the coding region of the decarbonylase gene derived from the N. punctiforme PCC 73102 strain.

An amino acid residue to be substituted is at least 1 amino acid residue selected from the group consisting of valine at position 29, glutamic acid at position 35, asparagine at position 39, threonine at position 42, histidine at position 51, leucine at position 54, methionine at position 60, serine at position 89, asparagine at position 94, leucine at position 169, asparagine at position 174, leucine at position 175, isoleucine at position 177, and aspartic acid at position 188 in the amino acid sequence as shown in SEQ ID NO: 2. Such amino acid residues to be substituted are positioned in the α helix structure constituting a decarbonylase.

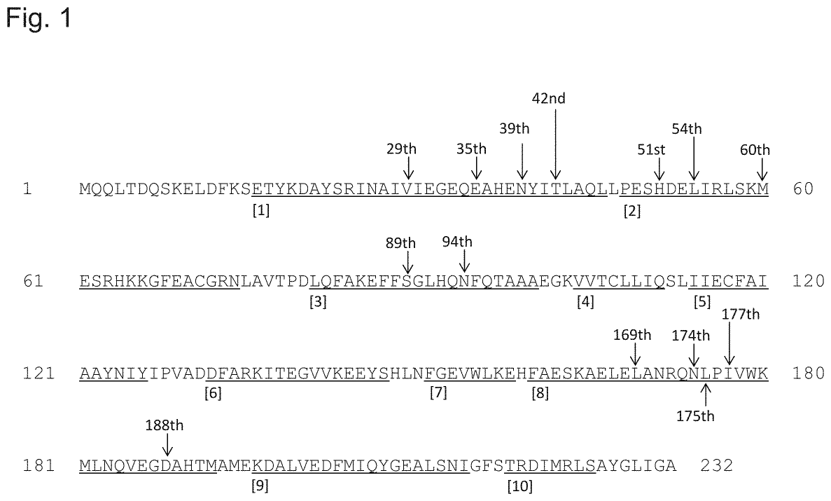

The decarbonylase derived from the N. punctiforme PCC 73102 strain is found to comprise 10 α helices as a result of the structural analysis based on the amino acid sequence thereof. Such 10 α helices are referred to as Helix 1 to Helix 10 sequentially from the N terminus. FIG. 1 shows the amino acid sequence of the decarbonylase derived from the N. punctiforme PCC 73102 strain (SEQ ID NO: 2) with numbering the 10 α helix structures (i.e., Helix 1 to Helix 10, underlined, the numbers are each in a circle). In FIG. 1, the amino acid residues to be substituted are indicated by arrows.

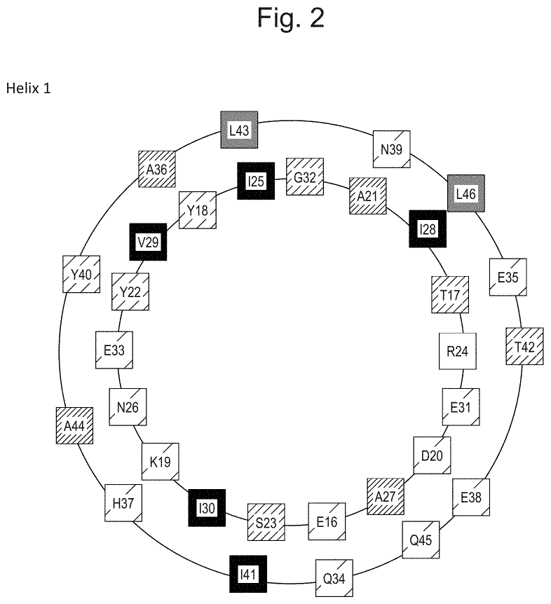

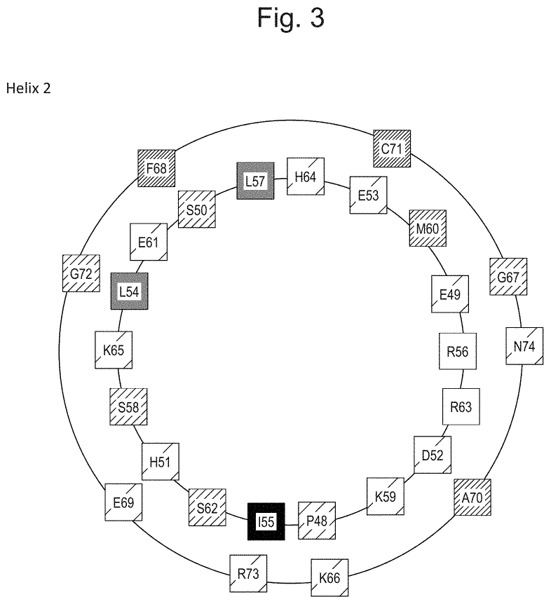

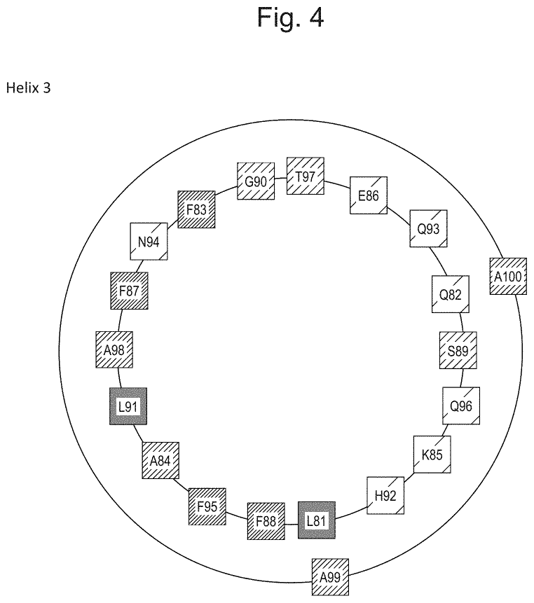

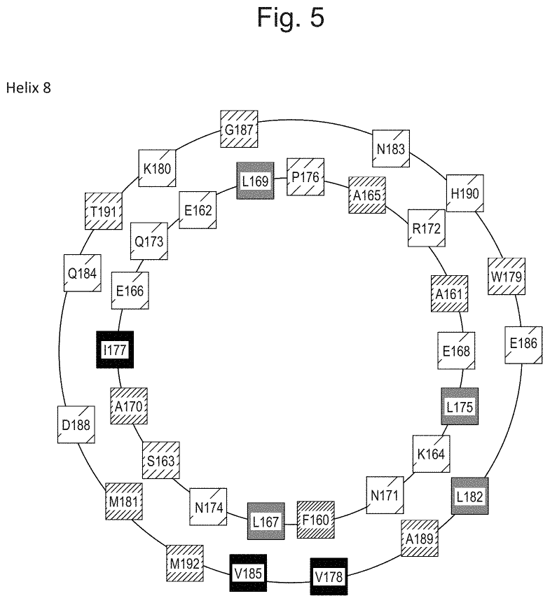

As shown in FIG. 1, amino acid residues to be substituted are located in Helix 1, Helix 2, Helix 3, and Helix 8. FIG. 2 to FIG. 5 each show amino acid sequences constituting the α helix structures; i.e., Helix 1, Helix 2, Helix 3, and Helix 8, arranged in circles, so as to observe the α helix structures in the axial direction. In FIG. 2 to FIG. 5, numbers following alphabetical letters representing amino acid types indicate the positions of amino acids when methionine at the N terminus is designated as “amino acid at position 1.” In FIG. 2, specifically, “V29” indicates valine at position 29 in the amino acid sequence as shown in SEQ ID NO: 2.

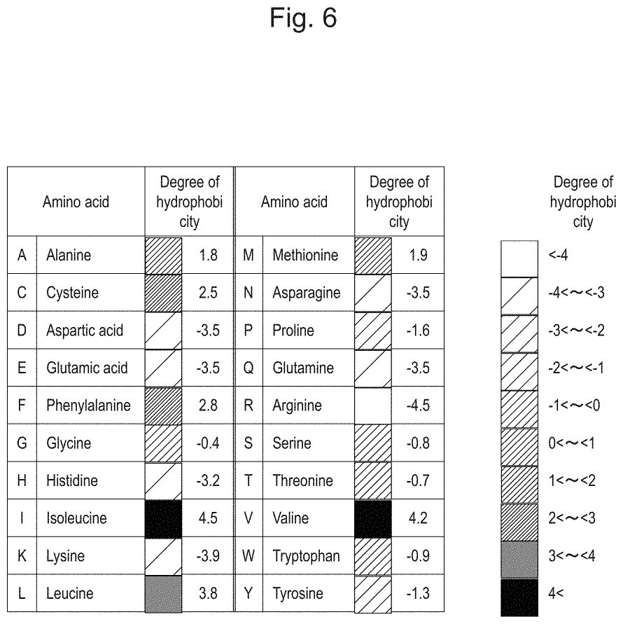

FIG. 2 to FIG. 5 each show amino acids superposed on the background patterns in accordance with the degrees of hydrophobicity. Specifically, as shown in FIG. 6, the degree of hydrophobicity described in Kyte J & Doolittle R F, 1982, J. Mol. Biol., 157: 105-132 was classified into 10 different levels, and each level was provided with a relevant background pattern. In this example, the background pattern was set to gradually increase the brightness as the degree of hydrophobicity increased. As shown in FIG. 2 to FIG. 5, amino acid sequences constituting the α helix structures are positioned sequentially in a circle, and the background pattern is set in accordance with the degree of hydrophobicity. Among amino acid residues arranged in the axial direction of the α helix structure, amino acid residues with different degrees of hydrophobicity can be visually and easily identified.

As shown in FIG. 2, for example, alanine at position 27, valine at position 29, and asparagine at position 39 exhibit the degree of hydrophobicity different from amino acid residues located in the vicinity thereof in Helix 1. More specifically, FIG. 2 demonstrates that the degree of hydrophobicity of alanine at position 27 and that of valine at position 29 are extremely higher than those of amino acids located in the vicinity thereof and the degree of hydrophobicity of asparagine at position 39 is extremely lower than those of amino acids located in the vicinity thereof. Further specifically, alanine at position 27, valine at position 29, asparagine at position 39, and the like in Helix 1 shown in FIG. 2 may be subjected to a substitution mutation, so as to adjust the degrees of hydrophobicity thereof to those of amino acids located in the vicinity thereof.

Based on the above, amino acids to be substituted in Helix 1 are valine at position 29, glutamic acid at position 35, asparagine at position 39, and threonine at position 42 as described in the examples below. When alanine at position 27 was substituted with an amino acid having the degree of hydrophobicity equivalent to that of amino acids in the vicinity thereof, decarbonylase activity was not improved. Accordingly, alanine at position 27 is not the amino acid to be substituted. Amino acids to be substituted in Helix 2 are histidine at position 51, leucine at position 54, and methionine at position 60 as described in the examples below. Amino acids to be substituted in Helix 3 are serine at position 89 and asparagine at position 94 as described in the examples below. Amino acids to be substituted in Helix 8 are leucine at position 169, asparagine at position 174, leucine at position 175, isoleucine at position 177, and aspartic acid 188.

Valine at position 29 included in Helix 1 has an extremely higher degree of hydrophobicity than amino acid residues in the vicinity thereof. In some embodiments, accordingly, it may be substituted with an amino acid with a low degree of hydrophobicity. Specifically, valine at position 29 may be substituted with an amino acid selected from the group consisting of tyrosine, tryptophan, serine, glycine, alanine, methionine, cysteine, phenylalanine, and leucine. In some other embodiments, valine at position 29 may be substituted with an amino acid selected from the group consisting of tyrosine, tryptophan, serine, glycine, alanine, and methionine. In some other embodiments, valine at position 29 may be substituted with methionine.

Glutamic acid at position 35 included in Helix 1 has an extremely lower degree of hydrophobicity than amino acid residues in the vicinity thereof. In some embodiments, accordingly, it may be substituted with an amino acid with a high degree of hydrophobicity. Specifically, glutamic acid at position 35 may be substituted with an amino acid selected from the group consisting of proline, tyrosine, tryptophan, serine, threonine, glycine, alanine, methionine, cysteine, phenylalanine, leucine, valine, and isoleucine. In some other embodiments, glutamic acid at position 35 may be substituted with an amino acid selected from the group consisting of proline, tyrosine, tryptophan, serine, threonine, and glycine. In some other embodiments, glutamic acid at position 35 may be substituted with tyrosine.

Asparagine at position 39 included in Helix 1 has an extremely lower degree of hydrophobicity than amino acid residues in the vicinity thereof. In some embodiments, accordingly, it may be substituted with an amino acid with a high degree of hydrophobicity. Specifically, asparagine at position 39 may be substituted with an amino acid selected from the group consisting of glycine, alanine, cysteine, phenylalanine, leucine, valine, and isoleucine. In some other embodiments, asparagine at position 39 may be substituted with an amino acid selected from the group consisting of cysteine, phenylalanine, leucine, valine, and isoleucine. In some other embodiments, asparagine at position 39 may be substituted with valine.

Threonine at position 42 included in Helix 1 has an extremely higher degree of hydrophobicity than amino acid residues in the vicinity thereof. In some embodiments, accordingly, it may be substituted with an amino acid with a low degree of hydrophobicity. Specifically, threonine at position 42 may be substituted with an amino acid selected from the group consisting of arginine, lysine, glutamine, asparagine, aspartic acid, glutamic acid, histidine, proline, tyrosine, tryptophan, serine, and glycine. In some other embodiments, threonine at position 42 may be substituted with an amino acid selected from the group consisting of lysine, glutamine, asparagine, aspartic acid, glutamic acid, histidine, proline, and tyrosine. In some other embodiments, threonine at position 42 may be substituted with asparagine or aspartic acid.

Histidine at position 51 included in Helix 2 has an extremely lower degree of hydrophobicity than amino acid residues in the vicinity thereof. In some embodiments, accordingly, it may be substituted with an amino acid with a high degree of hydrophobicity. Specifically, histidine at position 51 may be substituted with an amino acid selected from the group consisting of proline, tyrosine, tryptophan, serine, threonine, and glycine. In some other embodiments, histidine at position 51 may be substituted with proline or tyrosine. In some other embodiments, histidine at position 51 may be substituted with tyrosine.

Leucine at position 54 included in Helix 2 has an extremely higher degree of hydrophobicity than amino acid residues in the vicinity thereof. In some embodiments, accordingly, it may be substituted with an amino acid with a low degree of hydrophobicity. Specifically, leucine at position 54 may be substituted with an amino acid selected from the group consisting of glutamine, asparagine, aspartic acid, histidine, proline, tyrosine, tryptophan, serine, threonine, and glycine. In some other embodiments, leucine at position 54 may be substituted with an amino acid selected from the group consisting of glutamine, asparagine, aspartic acid, histidine, proline, and tyrosine. In some other embodiments, leucine at position 54 may be substituted with glutamine.

Methionine at position 60 included in Helix 2 has an extremely higher degree of hydrophobicity than amino acid residues in the vicinity thereof. In some embodiments, accordingly, it may be substituted with an amino acid with a low degree of hydrophobicity. Specifically, methionine at position 60 may be substituted with an amino acid selected from the group consisting of glutamine, aspartic acid, glutamic acid, histidine, proline, and tyrosine. In some other embodiments, methionine at position 60 may be substituted with an amino acid selected from the group consisting of glutamine, aspartic acid, and glutamic acid. In some other embodiments, methionine at position 60 may be substituted with aspartic acid.

Serine at position 89 included in Helix 3 has an extremely higher degree of hydrophobicity than amino acid residues in the vicinity thereof. In some embodiments, accordingly, it may be substituted with an amino acid with a low degree of hydrophobicity. Specifically, serine at position 89 may be substituted with an amino acid selected from the group consisting of glutamine, asparagine, aspartic acid, glutamic acid, histidine, proline, and tyrosine. In some other embodiments, serine at position 89 may be substituted with an amino acid selected from the group consisting of glutamine, asparagine, aspartic acid, and glutamic acid. In some other embodiments, serine at position 89 may be substituted with asparagine.

Asparagine at position 94 included in Helix 3 has an extremely lower degree of hydrophobicity than amino acid residues in the vicinity thereof. In some embodiments, accordingly, it may be substituted with an amino acid with a high degree of hydrophobicity. Specifically, asparagine at position 94 may be substituted with an amino acid selected from the group consisting of cysteine, phenylalanine, leucine, valine, and isoleucine. In some other embodiments, asparagine at position 94 may be substituted with an amino acid selected from the group consisting of leucine, valine, and isoleucine. In some other embodiments, asparagine at position 94 may be substituted with valine.

Leucine at position 169 included in Helix 8 has an extremely higher degree of hydrophobicity than amino acid residues in the vicinity thereof. In some embodiments, accordingly, it may be substituted with an amino acid with a low degree of hydrophobicity. Specifically, leucine at position 169 may be substituted with an amino acid selected from the group consisting of proline, tyrosine, tryptophan, serine, threonine, glycine, alanine, and methionine. In some other embodiments, leucine at position 169 may be substituted with an amino acid selected from the group consisting of tyrosine, tryptophan, serine, threonine, glycine, and alanine. In some other embodiments, leucine at position 169 may be substituted with an amino acid selected from the group consisting of tyrosine, tryptophan, and alanine.

Asparagine at position 174 included in Helix 8 has an extremely lower degree of hydrophobicity than amino acid residues in the vicinity thereof. In some embodiments, accordingly, it may be substituted with an amino acid with a high degree of hydrophobicity. Specifically, asparagine at position 174 may be substituted with an amino acid selected from the group consisting of proline, tyrosine, tryptophan, serine, threonine, glycine, alanine, methionine, cysteine, and phenylalanine. In some other embodiments, asparagine at position 174 may be substituted with an amino acid selected from the group consisting of tryptophan, serine, threonine, glycine, alanine, and methionine. In some other embodiments, asparagine at position 174 may be substituted with threonine or methionine.

Leucine at position 175 included in Helix 8 has an extremely higher degree of hydrophobicity than amino acid residues in the vicinity thereof. In some embodiments, accordingly, it may be substituted with an amino acid with a low degree of hydrophobicity. Specifically, leucine at position 175 may be substituted with an amino acid selected from the group consisting of arginine, lysine, glutamine, asparagine, aspartic acid, glutamic acid, histidine, proline, and tyrosine. In some other embodiments, leucine at position 175 may be substituted with an amino acid selected from the group consisting of lysine, glutamine, asparagine, aspartic acid, glutamic acid, and histidine. In some other embodiments, leucine at position 175 may be substituted with an amino acid selected from the group consisting of lysine, glutamine, and glutamic acid.

Isoleucine at position 177 included in Helix 8 has an extremely higher degree of hydrophobicity than amino acid residues in the vicinity thereof. In some embodiments, accordingly, it may be substituted with an amino acid with a low degree of hydrophobicity. Specifically, isoleucine at position 177 may be substituted with an amino acid selected from the group consisting of glutamine, asparagine, aspartic acid, glutamic acid, histidine, proline, tyrosine, tryptophan, serine, threonine, glycine, alanine, and methionine. In some other embodiments, isoleucine at position 177 may be substituted with an amino acid selected from the group consisting of proline, tyrosine, tryptophan, serine, threonine, and glycine. In some other embodiments, isoleucine at position 177 may be substituted with tyrosine or tryptophan.

Aspartic acid at position 188 included in Helix 8 has an extremely lower degree of hydrophobicity than amino acid residues in the vicinity thereof. In some embodiments, accordingly, it may be substituted with an amino acid with a high degree of hydrophobicity. Specifically, aspartic acid at position 188 may be substituted with an amino acid selected from the group consisting of cysteine, phenylalanine, leucine, valine, and isoleucine. In some other embodiments, aspartic acid at position 188 may be substituted with an amino acid selected from the group consisting of leucine, valine, and isoleucine. In some other embodiments, aspartic acid at position 188 may be substituted with valine.

As described above, a decarbonylase mutant resulting from a substitution mutation of a given amino acid residue exhibits decarbonylase activity superior to that of a decarbonylase without such mutation (e.g., a wild-type decarbonylase). Accordingly, recombinant microorganisms that express decarbonylase mutants would have the hydrocarbon-producing capacity superior to that of microorganisms expressing, for example, a decarbonylase comprising the amino acid sequence as shown in SEQ ID NO: 2.

The mutant decarbonylase gene described above is not limited to the gene encoding the decarbonylase mutant resulting from introduction of the above substitution mutation into the amino acid sequence as shown in SEQ ID NO: 2. It may be a gene encoding the decarbonylase mutant resulting from introduction of the above substitution mutation into an amino acid sequence different from the amino acid sequence as shown in SEQ ID NO: 2. While a detailed description is provided below, specific numerical values and amino acid types concerning the amino acid residues to be substituted are defined to be different from those concerning a decarbonylase comprising an amino acid sequence different from the amino acid sequence as shown in SEQ ID NO: 2.

An example of a decarbonylase comprising an amino acid sequence different from the amino acid sequence as shown in SEQ ID NO: 2 is a decarbonylase comprising an amino acid sequence exhibiting high similarity and/or identity to that of a wild-type decarbonylase encoded by the decarbonylase gene derived from the N. punctiforme PCC 73102 strain. A specific example thereof is a gene encoding a protein comprising an amino acid sequence exhibiting 50%, 60%, 70%, 80%, 85%, or 90% or higher identity to the amino acid sequence as shown in SEQ ID NO: 2 and having the decarbonylase activity as described above. Another specific example is a gene encoding a protein comprising an amino acid sequence exhibiting 80%, 85%, 90%, 95%, or 97% or higher similarity to the amino acid sequence as shown in SEQ ID NO: 2 and having the decarbonylase activity as described above.

The degree of sequence identity can be determined using the BLASTN or BLASTX Program equipped with the BLAST algorithm (at default settings). The degree of sequence identity is determined by subjecting a pair of amino acid sequences to pairwise alignment analysis, identifying completely identical amino acid residues, and calculating the percentage of all the amino acid residues subjected to comparison accounted for by such amino acid residues. The degree of sequence similarity is determined by subjecting a pair of amino acid sequences to pairwise alignment analysis, identifying completely identical amino acid residues and amino acid residues exhibiting similar functions, determining the total number of such amino acid residues, and calculating the percentage of all the amino acid residues subjected to comparison accounted for by the total number of such amino acid residues.

A decarbonylase comprising an amino acid sequence different from the amino acid sequence as shown in SEQ ID NO: 2 may be a protein comprising an amino acid sequence derived from the amino acid sequence as shown in SEQ ID NO: 2 by deletion, substitution, addition, or insertion of 1 to 50, 1 to 40, 1 to 30, or 1 to 20 amino acids and having decarbonylase activity.

A decarbonylase comprising an amino acid sequence different from the amino acid sequence as shown in SEQ ID NO: 2 may be a protein encoded by a nucleic acid hybridizing under stringent conditions to the full-length sequence or a partial sequence of a complementary strand of DNA comprising the nucleotide sequence as shown in SEQ ID NO: 1 and having decarbonylase activity. Under “stringent conditions,” so-called specific hybrids are formed, but non-specific hybrids are not formed. For example, such conditions can be adequately determined with reference to Molecular Cloning: A Laboratory Manual (Third Edition). Specifically, the degree of stringency can be determined in accordance with the temperature and the salt concentration of a solution used for Southern hybridization and the temperature and the salt concentration of a solution used for the step of washing in Southern hybridization.

A method for preparing DNA comprising a nucleotide sequence encoding a decarbonylase comprising an amino acid sequence different from the amino acid sequence as shown in SEQ ID NO: 2 or DNA comprising a nucleotide sequence different from the nucleotide sequence as shown in SEQ ID NO: 1 is not particularly limited, and a known method can be adequately adopted. For example, given nucleotides can be substituted in accordance with a site-directed mutagenesis technique. Examples of site-directed mutagenesis techniques include a method of site-directed mutagenesis (i.e., the Kunkel method, T. Kunkel, T. A., Proc. Nati. Acad. Sci., U.S.A., 82, 488-492, 1985) and the Gapped duplex method. Alternatively, a mutation can be introduced with the use of, for example, a mutagenesis kit that adopts a site-directed mutagenesis technique (e.g., Mutan-K and Mutan-G, manufactured by TAKARA SHUZO CO., LTD.) or an LA PCR in vitro Mutagenesis series kit manufactured by TAKARA SHUZO CO., LTD.

Table 1 shows a list of microorganisms comprising genes each encoding a decarbonylase comprising an amino acid sequence exhibiting high similarity and/or identity to the wild-type decarbonylase encoded by the decarbonylase gene derived from the N. punctiforme PCC 73102 strain.

TABLE 1

| Alkane- | | | | | |
|---|---|---|---|---|---|
| synthesizing | | Similarity | Identity | | GenBank |
| capacity | Organism | (%) | (%) | Gene | accession No. |
| | Nostoc sp. KVJ20 | 99.5 | 95.2 | A4S05_30645 | ODH01054 |
| | Anabaena cylindrica PCC 7122 | 98.2 | 87.0 | Anacy_3389 | AFZ58792 |
| | Anabaena azollae 0708 | 98.7 | 86.6 | Aazo_3371 | ADI65029 |
| | Nostoc sp. PCC 7524 | 97.4 | 86.1 | Nos7524_4304 | AFY50063 |
| | Calothrix sp. PCC 7507 | 99.1 | 86.1 | Cal7507_5586 | AFY35912 |
| | Anabaena sp. wa102 | 96.9 | 85.7 | AA650_00525 | ALB39141 |
| | Cylindrospermum stagnate PCC 7417 | 98.2 | 85.3 | Cylst_0697 | AFZ23025 |
| | Fischerella sp. NIES-3754 | 98.2 | 85.2 | FIS3754_06310 | BAU04742 |
| ◯ | Hapalosiphon welwitschii IC-52-3 | 98.2 | 85.2 | none | AHH34192 |
| ◯ | Westiella intricate HT-29-1 | 98.2 | 85.2 | none | AHH34193 |
| | Gloeocapsa sp. PCC 7428 | 97.4 | 84.9 | Glo7428_0150 | AFZ28764 |
| | Anabaena sp. 90 | 96.9 | 84.9 | ANA_C11210 | AFW93991 |
| | Nostoc sp. NIES-3756 | 96.5 | 83.9 | NOS3756_54760 | BAT56469 |
| | Microcoleus sp. PCC 7113 | 96.5 | 83.5 | Mic7113_4535 | AFZ20220 |
| | Chroococcidiopsis thermalis PCC 7203 | 97.4 | 82.6 | Chro_1554 | AFY87078 |
| | Calothrix sp. PCC 6303 | 97.4 | 82.6 | Cal6303_4369 | AFZ03276 |
| ◯ | Nostoc sp. PCC 7120 (Anabaena sp. | 97.8 | 82.6 | alr5283 | BAB76982 |
| | PCC 7120) | | | | |
| | Nostoc sp. PCC 7107 | 95.6 | 82.2 | Nos7107_1028 | AFY41687 |
| | Calothrix sp. 336_3 | 97.4 | 81.8 | IJ00_07390 | AKG21145 |
| | Nostoc punctiforme PCC73102 | 97.4 | 81.3 | Npun_R1711 | ACC80382 |
| | Crinalium epipsammum PCC 9333 | 96.9 | 81.2 | Cri9333_4418 | AFZ15201 |
| | Cyanothece sp. PCC 8802 | 96.5 | 80.5 | none | Cyan8802_0468(KEGG)* |
| | Cyanothece sp. PCC 8801 | 96.5 | 80.5 | PCC8801_0455 | ACK64551 |
| | Rivularia sp. PCC 7116 | 97.4 | 80.5 | Riv7116_3790 | AFY56233 |
| | Oscillatoria acuminata PCC 6304 | 96.1 | 79.7 | Oscil6304_2075 | AFY81740 |
| | Cyanothece sp. ATCC 51142 | 96.1 | 77.9 | cce_0778 | ACB50129 |
| | Arthrospira platensis NIES-39 | 95.2 | 77.9 | NIES39_M01940 | BAI93031 |
| ◯ | Gloeobacter violaceus PCC 7421 | 96.1 | 77.9 | gll3146 | BAC91087 |
| | Oscillatoria nigro-viridis PCC 7112 | 97.3 | 77.8 | Osc7112_0944 | AFZ05510 |
| ◯ | Oscillatoria sp. PCC 6506 | 96.1 | 77.4 | OSCI_940017 | CBN54532 |
| | Dactylococcopsis salina PCC 8305 | 96.1 | 77.0 | Dacsa_2178 | AFZ50804 |
| | Chamaesiphon minutus PCC 6605 | 93.9 | 76.6 | Cha6605_4153 | AFY95099 |
| | Leptolyngbya sp. 0-77 | 94.8 | 76.1 | O77CONTIG1_03123 | BAU43295 |
| | Trichodesmium erythraeum IMS101 | 96.1 | 75.8 | Tery_2280 | ABG51506 |
| | Pseudanabaena sp. PCC 7367 | 93.5 | 75.3 | Pse7367_3626 | AFY71859 |
| ◯ | Planktothrix agardhii NIV-CYA | 94.3 | 75.2 | A19Y_4321 | KEI68998 |
| | Leptolyngbya boryana IAM M-101 | 96.5 | 74.8 | LBWT_14420 | LBWT_14420(KEGG)* |
| | Leptolyngbya sp. NIES-3755 | 96.5 | 74.8 | LEP3755_23570 | BAU11854 |
| | Halothece sp. PCC 7418 | 95.6 | 74.4 | PCC7418_0961 | AFZ43170 |
| | Acaryochloris marina MBIC11017 | 92.6 | 74.4 | AM1_4041 | ABW29023 |
| | Microcystis panniformis FACHB-1757 | 93.5 | 74.4 | VL20_1523 | AKV66681 |
| | Synechocystis sp. PCC 6714 | 95.6 | 73.5 | D082_05310 | AIE73060 |
| | Candidatus Atelocyanobacterium thalassa | 93.9 | 73.5 | ucyna2_01151 | KFF41020 |
| | Synechocystis sp. PCC 6803 PCC-P | 95.6 | 73.2 | sll0208 | SYNPCCP_2250(KEGG)* |
| | Synechocystis sp. PCC 6803 PCC-N | 95.6 | 73.1 | sll0208 | SYNPCCN_2250(KEGG)* |
| | Synechocystis sp. PCC 6803 GT-I | 95.6 | 73.1 | sll0208 | SYNGTI_2251(KEGG)* |
| | Microcystis aeruginosa NIES-843 | 93 | 73.1 | MAE_53090 | BAG05131 |
| ◯ | Synechocystis sp. PCC 6803 | 95.6 | 73.1 | sll0208 | BAA10217 |
| | Thermosynechococcus sp. NK55 | 93.9 | 72.7 | NK55_03185 | AHB87984 |
| | Synechococcus sp. UTEX 2973 | 93 | 72.7 | M744_09020 | M744_09020(KEGG)* |
| | Synechococcus elongatus PCC6301 | 93 | 72.7 | syc0050_d | BAD78240 |
| ◯ | Synechococcus elongatus PCC7942 | 93 | 72.7 | Synpcc7942_1593 | ABB57623 |
| ◯ | Thermosynechococcus elongatus BP-1 | 94.3 | 72.7 | tll1313 | BAC08865 |
| | Synechococcus sp. PCC 7502 | 95.2 | 72.4 | Syn7502_03278 | AFY75144 |
| | Synechococcus sp. PCC 6312 | 96 | 71.7 | Syn6312_2280 | AYF64395 |
| | Geminocystis sp. NIES-3708 | 93.4 | 71.7 | GM3708_2118 | BAQ61712 |
| | Cyanobacterium aponinum PCC 10605 | 93.4 | 70.8 | Cyan10605_1692 | AFZ53795 |
| ◯ | Cyanothece sp. PCC 7425 | 96.1 | 70.5 | Cyan7425_0398 | ACL42790 |
| ◯ | Anabaena variabilis ATCC 29413 | 96.1 | 70.5 | Ava_2533 | ABA22148 |
| | Cyanobacterium endosymbiont of | 93.5 | 70.1 | ETSB_0877 | BAP17683 |
| | Epithemia turgida | | | | |
| | Synechococcus sp. JA-2-3B′a(2-13) | 92.6 | 66.3 | CYB_2442 | ABD03376 |
| ◯ | Synechococcus sp. JA-3-3Ab | 91.8 | 65.0 | CYA_0415 | ABC98634 |
| ◯ | Synechocystis sp. RS9917 | 90 | 63.6 | RS9917_09941 | EAQ69748 |
| | Gloeobacter kilaueensis JS1 | 90.5 | 62.9 | GKIL_0725 | AGY56971 |
| | Synechococcus sp. WH7803 | 86.5 | 62.7 | SynWH7803_0654 | CAK23080 |
| | Cyanobium gracile PCC 6307 | 89.1 | 61.9 | Cyagr_0039 | AFY27259 |
| | Synechococcus sp. KORDI-52 | 88.7 | 61.9 | KR52_13300 | AII50102 |
| | Synechococcus sp. WH 8109 | 88.7 | 61.4 | Syncc8109_1976 | AHF64320 |
| | Synechococcus sp. CC9605 | 88.7 | 61.4 | Syncc9605_0728 | ABB34500 |
| | Synechococcus sp. KORDI-49 | 88.3 | 61.0 | KR49_12745 | AII47259 |
| | Synechococcus sp. CC9902 | 88.3 | 61.0 | Syncc9902_1635 | ABB26593 |
| | Synechococcus sp. KORDI-100 | 89.6 | 60.6 | KR100_05365 | AII42794 |
| | Synechococcus sp. WH8102 | 88.3 | 60.1 | SYNW1738 | CAE08253 |
| | Synechococcus sp. RCC307 | 88.3 | 59.7 | SynRCC307_1586 | CAK28489 |
| | Prochlorococcus marinus MIT 9303 | 90 | 59.3 | P9303_07791 | ABM77530 |
| | Synechococcus sp. CC9311 | 88.3 | 59.3 | sync_1990 | ABI47589 |
| ◯ | Prochlorococcus marinus MIT 9313 | 89.6 | 58.8 | PMT_1231 | CAE21406 |
| | Cyanothece sp. PCC 7425 | 90.4 | 57.5 | Cyan7425_2986 | ACL45322 |
| | Prochlorococcus marinus MED4 | 88.2 | 56.5 | PMM0532 | CAE18991 |
| | Prochlorococcus marinus MIT 9515 | 87.8 | 55.6 | P9515_05961 | ABM71805 |
| | Prochlorococcus marinus MIT 9301 | 86.9 | 55.2 | P9301_05581 | ABO1718 |
| | Prochlorococcus marinus AS9601 | 87.3 | 55.2 | A9601_05881 | ABM69874 |
| | Prochlorococcus marinus MIT 9215 | 87.3 | 55.2 | P9215_06131 | ABV50228 |
| | Prochlorococcus marinus MIT 9312 | 87.3 | 54.7 | PMT9312_0532 | ABB49593 |
| | Prochlorococcus sp. MIT 0604 | 87.3 | 54.3 | EW14_0578 | AIQ94601 |
| | Prochlorococcus marinus MIT 9211 | 88.6 | 53.9 | P9211_05351 | ABX08466 |
| | Prochlorococcus marinus NATL1A | 87.8 | 53.4 | NATL1_05881 | ABM75150 |
| ◯ | Prochlorococcus marinus NATL2A | 87.8 | 53.4 | PMN2A_1863 | AAZ59351 |
| | Prochlorococcus sp. MIT 0801 | 88.2 | 53.0 | EW15_0629 | AIQ96721 |
| | Prochlorococcus marinus SS120 | 87.8 | 51.3 | Pro_0532 | AAP99577 |

(KEGG)*: KEGG entry number

In Table 1, microorganisms indicated with the symbol “∘” in the “alkane-synthesizing capacity” column were reported to have the alkane-synthesizing capacity. The nucleotide sequences of the coding regions of the decarbonylase genes of the microorganisms shown in Table 1 and the amino acid sequences encoded thereby can be obtained from the GenBank database or other databases on the basis of the names and the GenBank accession numbers shown in Table 1.

Concerning the decarbonylases derived from the microorganisms shown in Table 1, the amino acid sequences obtained from the database and the amino acid sequence as shown in SEQ ID NO: 2 are subjected to pairwise alignment analysis. Thus, the amino acid residues to be substituted can be identified. Among the amino acid residues to be substituted, for example, valine at position 29 in the amino acid sequence as shown in SEQ ID NO: 2 may not be located in the position 29 in an amino acid sequence different from the amino acid sequence as shown in SEQ ID NO: 2. In addition, an amino acid residue in the corresponding position may be an amino acid other than valine. In such a case, an amino acid residue in an amino acid sequence different from the amino acid sequence as shown in SEQ ID NO: 2, which corresponds to valine at position 29 in the amino acid sequence as shown in SEQ ID NO: 2, is to be substituted. When an expression such as “an amino acid corresponding to valine at position 29” is used herein, such expression encompasses both valine at position 29 in the amino acid sequence as shown in SEQ ID NO: 2 and an amino acid corresponding to valine 29 in an amino acid sequence different from the amino acid sequence as shown in SEQ ID NO: 2.

As described above, amino acids to be substituted in the amino acid sequence as shown in SEQ ID NO: 2 are valine at position 29, glutamic acid at position 35, asparagine at position 39, threonine at position 42, histidine at position 51, leucine at position 54, methionine at position 60, serine at position 89, asparagine at position 94, leucine at position 169, asparagine at position 174, leucine at position 175, isoleucine at position 177, and aspartic acid at position 188. In an amino acid sequence different from the amino acid sequence as shown in SEQ ID NO: 2, specifically, amino acid residues corresponding to such specific amino acid residues are to be substituted.

In an amino acid sequence different from the amino acid sequence as shown in SEQ ID NO: 2, the amino acid residues after the substitution would be the same in the case of the decarbonylase comprising the amino acid sequence as shown in SEQ ID NO: 2. As shown in Table 1, this is because that an amino acid sequence different from the amino acid sequence as shown in SEQ ID NO: 2 shows very high identity to the amino acid sequence as shown in SEQ ID NO: 2.

There are 4 other examples of decarbonylase genes encoding a decarbonylase: (1) decarbonylases typified by Npun_R1711 of Nostoc punctiforme (Science mentioned above); (2) a decarbonylase related to an aldehyde dehydrogenase (JP Patent No. 5,867,586); (3) long-chain alkane synthases typified by the Cer1 gene of Arabidopsis thaliana (Plant Cell, 24, 3106-3118, 2012); and (4) P450 alkane synthases typified by the CYP4G1 gene of Drosophila melanogaster (PNAS, 109, 37, 14858-14863, 2012).

More specific examples of (1) include Npun_R0380 of Nostoc punctiforme (a paralog of Npun_R1711), Nos7524_4304 of Nostoc sp., Anacy_3389 of Anabaena cylindrica, Aazo_3371 of Anabaena azollae, Cylst_0697 of Cylindrospermum stagnale, Glo7428_0150 of Gloeocapsa sp., Ca17507_5586 of Calothrix sp., FIS3754_06310 of Fischerella sp., Mic7113_4535 of Microcoleus sp., Chro_1554 of Chroococcidiopsis thermalis, GEI7407_1564 of Geitlerinema sp., and Cyan8802_0468 of Cyanothece sp.

Specific examples of (2) include: BAE77705, BAA35791, BAA14869, BAA14992, BAA15032, BAA16524, BAE77705, BAA15538, and BAA15073 derived from Escherichia coli K-12 W3110; YP_001268218, YP_001265586, YP_001267408, YP_001267629, YP_001266090, YP_001270490, YP_001268439, YP_001267367, YP_001267724, YP_001269548, YP_001268395, YP_001265936, YP_001270470, YP_001266779, and YP_001270298 derived from Pseudomonas putida_F1; NP_388129, NP_389813, NP_390984, NP_388203, NP_388616, NP_391658, NP_391762, NP_391865, and NP_391675 derived from Bacillus subtilis 168; NP_599351, NP_599725, NP_601988, NP_599302, NP_601867, and NP_601908 derived from Corynebacterium glutamicum ATCC13032; YP_001270647 derived from Lactobacillus reuteri DSM20016; NP_010996, NP_011904, NP 015264, NP 013828, NP_009560, NP 015019, NP_013893, NP_013892, and NP_011902 derived from Saccharomyces cerevisiae; XP_002548035, XP_002545751, XP_002547036, XP_002547030, XP_002550712, XP_002547024, XP_002550173, XP_002546610, and XP_002550289 derived from Candida tropicalis MYA-3404; XP_460395, XP_457244, XP_457404, XP_457750, XP_461954, XP_462433, XP_461708, and XP_462528 derived from Debaryomyces hansenii CBS767; XP_002489360, XP_002493450, XP_002491418, XP_002493229, XP_002490175, XP_002491360, and XP_002491779 derived from Pichia pastoris GS115; NP_593172, NP_593499, and NP_594582 derived from Schizosaccharomyces pombe; XP_001822148, XP_001821214, XP_001826612, XP_001817160, XP_001817372, XP_001727192, XP_001826641, XP_001827501, XP_001825957, XP_001822309, XP_001727308, XP_001818713, XP_001819060, XP_001823047, XP_001817717, and XP_001821011 derived from Aspergillus oryzae RIB40; NP_001150417, NP_001105047, NP_001147173, NP_001169123, NP_001105781, NP_001157807, NP_001157804, NP_001105891, NP_001105046, NP_001105576, NP_001105589, NP_001168661, NP_001149126, and NP_001148092 derived from Zea mays; NP_564204, NP_001185399, NP_178062, NP_001189589, NP_566749, NP 190383, NP_187321, NP_190400, NP_001077676, and NP_175812 derived from Arabidopsis thaliana; NP_733183, NP_609285, NP_001014665, NP_649099, NP_001189159, NP_610285, and NP_610107 derived from Drosophila melanogaster; NP 001006999, XP_001067816, XP_001068348, XP_001068253, NP_113919, XP_001062926, NP_071609, NP_071852, NP_058968, NP_001011975, NP_115792, NP_001178017, NP_001178707, NP_446348, NP_071992, XP_001059375, XP_001061872, and NP_001128170 derived from Rattus norvegicus; NP_036322, NP_001193826, NP_001029345, NP_000684, NP_000680, NP_000683, NP_000681, NP_001071, NP_000687, NP_001180409, NP_001173, NP_000682, NP_000373, NP_001154976, NP_000685, and NP_000686 derived from Homo sapiens; and KPN_02991, KPN_1455, and KPN_4772 derived from Klebsiella sp. NBRC100048.

Specific examples of (3) include: AT1G02190 and AT1G02205 (CER1) of Arabidopsis thaliana; 4330012 of Oryza sativa; 101252060 of Solanum lycopersicum; CARUB_v10008547 mg of Capsella rubella; 106437024 of Brassica napus; 103843834 of Brassica rapa; EUTSA_v10009534 mg of Eutrema salsugineum; 104810724 of Tarenaya hassleriana; 105773703 of Gossypium raimondii; TCM_042351 of Theobroma cacao; 100243849 of Vitis vinifera; 105167221 of Sesamum indicum; 104442848 of Eucalyptus grandis; 103929751 of Pyrus bretschneideri; 107618742 of Arachis ipaensis; and 103428452 of Malus domestica.

Specific examples of (4) include CYP4G1 of Drosophila melanogaster, 101887882 of Musca domestica, AaeL_AAEL006824 of Aedes aegypti, and AgaP_AGAP000877 of Anopheles gambiae.

The various types of decarbonylase genes described above can be mutant decarbonylase genes each encoding a decarbonylase mutant comprising an amino acid sequence derived from the amino acid sequence as shown in SEQ ID NO: 2 by the substitution mutation defined above. Also, the mutant decarbonylase genes derived from the various types of decarbonylase genes described above each encode a decarbonylase mutant with enhanced decarbonylase activity.

As described above, the mutant decarbonylase gene according to the present disclosure is introduced into a host microorganism together with the acyl-ACP reductase gene that catalyzes conversion of acyl-ACP into fatty aldehyde or it is introduced into a host microorganism comprising the acyl-ACP reductase gene. Thus, a recombinant microorganism having the alkane-producing capacity can be prepared.

An acyl-ACP reductase gene is not particularly limited, and a gene encoding the acyl-ACP reductase registered as EC 1.2.1.80 can be used. Examples of acyl-ACP reductase genes include Synpcc7942_1594 of Synechococcus elongatus, M744_09025 of Synechococcus sp., LEP3755_23580 of Leptolyngbya sp., Glo7428_0151 of Gloeocapsa sp., Nos7107_1027 of Nostoc sp., Ava_2534 of Anabaena variabilis, IJ00_07395 of Calothrix sp., Cri9333_4415 of Crinalium epipsammum, and FIS3754_06320 of Fischerella sp.

For example, the acyl-ACP reductase gene derived from Synechococcus elongatus PCC 7942 encodes a protein comprising the amino acid sequence as shown in SEQ ID NO: 4. The acyl-ACP reductase gene may be a gene encoding a protein comprise an amino acid sequence exhibiting 60%, 70%, 80%, 90%, 95%, or 98% or higher identity to the amino acid sequence as shown in SEQ ID NO: 4 and having acyl-ACP reductase activity.

The degree of sequence identity can be determined using the BLASTN or BLASTX Program equipped with the BLAST algorithm (at default settings). The degree of sequence identity is determined by subjecting a pair of amino acid sequences to pairwise alignment analysis, identifying completely identical amino acid residues, and calculating the percentage of all the amino acid residues subjected to comparison accounted for by such amino acid residues.

The acyl-ACP reductase gene is not limited to a gene encoding the amino acid sequence as shown in SEQ ID NO: 4. It may be a gene encoding a protein comprising an amino acid sequence derived from the amino acid sequence as shown in SEQ ID NO: 4 by deletion, substitution, addition, or insertion of 1 to 50, 1 to 40, 1 to 30, or 1 to 20 amino acids and functioning as an acyl-ACP reductase.

Furthermore, the acyl-ACP reductase gene is not limited to a gene comprising the nucleotide sequence as shown in SEQ ID NO: 3. For example, it may be a gene hybridizing under stringent conditions to the full-length sequence or a partial sequence of a complementary strand of DNA comprising the nucleotide sequence as shown in SEQ ID NO: 3 and encoding a protein functioning as an acyl-ACP reductase. Under “stringent conditions,” so-called specific hybrids are formed, but non-specific hybrids are not formed. For example, such conditions can be adequately determined with reference to Molecular Cloning: A Laboratory Manual (Third Edition). Specifically, the degree of stringency can be determined in accordance with the temperature and the salt concentration of a solution used for Southern hybridization and the temperature and the salt concentration of a solution used for the step of washing in Southern hybridization.

A method for preparing DNA comprising a nucleotide sequence encoding an amino acid sequence derived from the amino acid sequence as shown in SEQ ID NO: 4 by deletion, substitution, addition, or insertion of given amino acids or DNA comprising a nucleotide sequence different from the nucleotide sequence as shown in SEQ ID NO: 3 is not particularly limited, and a known method can be adequately adopted. For example, given nucleotides can be substituted by a site-directed mutagenesis technique. Examples of site-directed mutagenesis techniques include a method of site-directed mutagenesis (i.e., the Kunkel method, T. Kunkel, T. A., Proc. Nati. Acad. Sci., U.S.A., 82, 488-492, 1985) and the Gapped duplex method. Alternatively, a mutation can be introduced with the use of, for example, a mutagenesis kit that adopts a site-directed mutagenesis technique (e.g., Mutan-K and Mutan-G, manufactured by TAKARA SHUZO CO., LTD.) or an LA PCR in vitro Mutagenesis series kit manufactured by TAKARA SHUZO CO., LTD.

In place of the acyl-ACP reductase gene, a gene encoding an enzyme that synthesizes aldehyde serving as a substrate for the decarbonylase mutant can be used.

For example, a gene encoding a long chain fatty acyl-CoA reductase (EC.1.2.1.50), such as plu2079 (luxC) of Photorhabdus luminescens, PAU_02514 (luxC) of Photorhabdus asymbiotica, VF_A0923 (luxC) of Aliivibrio fischeri, VIBHAR_06244 of Vibrio campbellii, or Swoo_3633 of Shewanella woodyi, can be used. Also, genes encoding acyl-CoA reductases described in JP 2015-226477 A, such as 100776505 and 100801815 of Glycine max, can be used. In addition, any gene encoding an enzyme that can synthesize an aldehyde can be used without particular limitation. For example, genes encoding enzymes, such as alcohol dehydrogenase (EC.1.1.1.1), alcohol oxidase (EC. 1.1.3.13), aldehyde dehydrogenase (EC. 1.2.1.3), and carboxylate reductase (EC. 1.2.99.6), can be used.

Microorganisms into which the mutant decarbonylase gene is to be introduced are not particularly limited, and examples include Escherichia coli and bacteria of the genera Klebsiella. As microorganisms into which the mutant decarbonylase gene is to be introduced, Corynebacterium glutamicum disclosed in Appl. Environ. Microbiol., 79 (21): 6776-6783, 2013 (November) can be used. This literature discloses a recombinant Corynebacterium glutamicum that has acquired the fatty acid-producing capacity. As microorganisms into which the mutant decarbonylase gene is to be introduced, in addition, Mortierella alpina disclosed in Food Bioprocess Technol., 2011, 4: 232-240 can be used. Mortierella alpina is used at the industrial level for arachidonic acid fermentation, and, in this literature, metabolic engineering is practiced with the use thereof. In addition, Yarrowia lipolytica disclosed in TRENDS IN BIOTECHNOLOGY, Vol. 34, No. 10, pp. 798-809 can be used as a microorganism into which the mutant decarbonylase gene is to be introduced.

As microorganisms into which the mutant decarbonylase gene is to be introduced, microorganisms belonging to the genera Lipomyces, Pseudozyma, Rhodosporidium, and Rhodococcus can be used. In order to introduce the alkane synthase gene into such microorganisms, a gene recombination technique involving the genome editing system, such as CRISPR/Cas or TALEN, can be adopted without particular limitation.

When yeast strains are used as microorganisms into which the mutant decarbonylase gene is to be introduced, examples of yeast strains that can be used include, but are not particularly limited to, a yeast strain that belongs to the genus Pichia such as Pichia stipitis, a yeast strain that belongs to the genus Saccharomyces such as Saccharomyces cerevisiae, and yeast strains that belong to the genus Candida such as Candida tropicalis and Candida prapsilosis.

When the mutant decarbonylase gene, the acyl-ACP reductase gene, and other genes are introduced into hosts, for example, a DNA fragment containing the mutant decarbonylase gene or the acyl-ACP reductase gene may be ligated to an expression vector that can function in a host microorganism (e.g., a multiple-copy vector) to prepare recombinant DNA, and the resulting recombinant DNA may then be introduced into a microorganism to transform the microorganism. Expression vectors that can be used are not particularly limited, and a plasmid vector or a chromosome transfer vector that can be incorporated into the genome of the host organism can be used. An expression vector is not particularly limited, and an available expression vector may be adequately selected in accordance with a host microorganism. Examples of expression vectors include plasmid DNA, bacteriophage DNA, retrotransposon DNA, and yeast artificial chromosome (YAC) DNA.

Examples of plasmid DNA include: YCp-type E. coli-yeast shuttle vectors, such as pRS413, pRS414, pRS415, pRS416, YCp50, pAUR112, and pAUR123; YEp-type E. coli-yeast shuttle vectors, such as pYES2 and YEpl3; YIp-type E. coli-yeast shuttle vectors, such as pRS403, pRS404, pRS405, pRS406, pAUR101, and pAUR135; E. coli-derived plasmids (e.g., ColE plasmids, such as pBR322, pBR325, pUC18, pUC19, pUC118, pUC119, pTV118N, pTV119N, pBluescript, pHSG298, pHSG396, and pTrc99A, p15A plasmids, such as pACYC177 and pACYC184, and pSC101 plasmids, such as pMW118, pMW119, pMW218, and pMW219); Agrobacterium-derived plasmids (e.g., pBI101); and Bacillus subtilis-derived plasmids (e.g., pUB110 and pTP5). Examples of phage DNA include λ phage (e.g., Charon4A, Charon21A, EMBL3, EMBL4, λgt10, λgt11, and λZAP), φX174, M13mp18, and M13mp19. An example of retrotransposon is a Ty factor. An example of a YAC vector is pYACC2. In addition, animal virus vectors, such as retrovirus or vaccinia virus vectors, and insect virus vectors, such as baculovirus vectors, can be used.

It is necessary that the mutant decarbonylase gene be incorporated into an expression vector in an expressible state. In an expressible state, the mutant decarbonylase gene is linked to a promoter, and the resultant is incorporated into a vector in that state, so that the mutant decarbonylase gene is expressed under the control of a given promoter in a host organism. In addition to the mutant decarbonylase gene, a promoter, a terminator, a cis element such as an enhancer according to need, a splicing signal, a poly A addition signal, a selection marker, a ribosome binding sequence (SD sequence), and the like can be linked to the expression vector. Examples of selection markers include antibiotic resistant genes, such as ampicillin resistant gene, kanamycin resistant gene, and hygromycin resistant gene.

As a method of transformation involving the use of an expression vector, a conventional technique can be adequately employed. Examples of methods of transformation include the calcium chloride method, the competent cell method, the protoplast or spheroplast method, and the electropulse method.

Alternatively, the mutant decarbonylase gene may be introduced to increase the number of copies thereof. Specifically, the mutant decarbonylase gene may be introduced in a manner such that multiple copies of the mutant decarbonylase genes would be present in chromosome DNA of the microorganism. Multiple copies of the mutant decarbonylase genes can be introduced into chromosome DNA of the microorganism via homologous recombination with the use of multiple copies of target sequences that are present in chromosome DNA.

The mutant decarbonylase gene expression level can be elevated by, for example, a method in which an expression regulatory sequence such as a promoter of the introduced mutant decarbonylase gene is substituted with a sequence that can express the gene of interest at a higher level or a method in which a regulator to elevate the expression level of a given gene is introduced. Examples of promoters that enable high level gene expression include, but are not particularly limited to, lac promoter, trp promoter, trc promoter, and pL promoter. Alternatively, a mutation may be introduced into an expression regulatory region of the endogenous or introduced ferredoxin gene or the ferredoxin reductase gene to modify the gene to be expressed at a higher level.

<Alkane Production>

As described above, alkane can be synthesized with excellent productivity with the use of a recombinant microorganism into which the mutant decarbonylase gene has been introduced.

In a system involving the use of recombinant microorganisms comprising the mutant decarbonylase gene introduced thereinto, culture can be conducted in a medium suitable for such microorganisms, and alkane can be produced in the medium. According to the present disclosure, more specifically, the alkane-synthesizing capacity with the aid of an alkane synthase can be improved, and alkane productivity can be improved as a consequence.

According to the present disclosure, alkane to be produced may have, for example, 9 to 20, 14 to 17, or 13 to 16 carbon atoms, although the number of carbon atoms is not limited thereto. These are liquid with high viscosity, and it can be used for light oil (diesel oil) or aircraft fuel. Such alkane can be isolated from a reaction system in which the recombinant microorganisms were cultured in accordance with a conventional technique and then purified. By adopting the method described in Engineering in Life Sciences, vol. 16:1, pp. 53-59, “Biosynthesis of chain-specific alkanes by metabolic engineering in Escherichia coli,” short-chain alkane can be synthesized.

### EXAMPLES

Hereafter, the present disclosure is described in greater detail with reference to the examples, although the technical scope of the present disclosure is not limited to the following examples.

### Example 1

[1. Objective]

A decarbonylase is a key enzyme used when producing alkane (hydrocarbon), which is a next-generation biodiesel fuel, via fermentation with the aid of microorganisms such as Escherichia coli. In order to develop a technique of enhancing enzyme activity of a decarbonylase, in this example, a substitution mutation of amino acids for α helix stabilization was introduced into a decarbonylase to prepare a decarbonylase mutant, and the substitution mutation of amino acids that would enhance decarbonylase activity was identified.

[2. Materials and Method]

2.1: Reagent

The plasmids used in the example: i.e., pRSF-Duet-1 and pCDF-Duet-1, were purchased from Novagen. In this example, reagents without the manufacturers thereof being specified were purchased from Nacalai tesque.

2.2: Strains

In this example, E. coli BL-21 purchased from Takara Bio Inc. and E. coli JM109 purchased from Nippon Gene Co., Ltd. were used.

2.3: Preparation of Plasmids

2.3.1: Preparation of pRSF-NpAD-PA

At the outset, pRSF-NpAD-SeAR was prepared in the manner described below. Specifically, the acyl-ACP reductase gene derived from Synechococcus elongatus PCC 7942 (YP_400611) and the decarbonylase gene derived from Nostoc punctiforme PCC 73102 (YP_001865325) were chemically synthesized. These synthetic genes were inserted into the EcoRV site of pUC57 and designated as pUC57-SeAAR and pUC57-NpAD, respectively.

Subsequently, pUC57-NpAD and pUC57-SeAAR were used as templates to perform PCR with the use of Pfu Ultra II Fusion HS DNA Polymerase (STRATAGENE) in the manner described below, and the amplified fragments; i.e., NpADvo and SeAAvo, were obtained.

TABLE 2
Reaction composition:

| | | |
|---|---|---|
| pUC57-NpAD (30 ng/μl) | 1 | μl |
| 10x Pfu Ultra II reaction buffer | 5 | μl |
| dNTP mix (25 mM each) | 1 | μl |
| Primer pRSF-NpAS-inf-F (10 μM) | 2 | μl |
| Primer pRSF-NpAS-inf-R (10 μM) | 2 | μl |
| Pfu Ultra II fusion HS DNA polymerase | 1 | μl |
| Sterilized deionized water | 38 | μl |
| Total | 50 | μl |

TABLE 3
Reaction composition:

| | | |
|---|---|---|
| pUC57-SeAAR (1 ng/μl) | 1 | μl |
| 10x Pfu Ultra II reaction buffer | 5 | μl |
| dNTP mix (25 mM each) | 1 | μl |
| Primer pRSF-SeAR-inf-F (10 μM) | 2 | μl |
| Primer pRSF-SeAR-inf-R (10 μM) | 2 | μl |
| Pfu Ultra II fusion HS DNA polymerase | 1 | μl |
| Sterilized deionized water | 38 | μl |
| Total | 50 | μl |

PCR temperature conditions comprises: 92° C. for 2 minutes, a cycle of 92° C. for 10 seconds, 55° for 20 seconds, and 68° C. for 5 minutes repeated 25 times, 72° C. for 3 minutes, and 16° C. Primer sequences are as shown below.

Primer pRSF-NpAS-inf-F:
(SEQ ID NO: 5)
5′-cgagctcggcgcgcctgcagATGCAGCAGCTTACAGACCA-3′
Primer pRSF-NpAS-inf-R:
(SEQ ID NO: 6)
5′-gcaagcttgtcgacctgcagTTAAGCACCTATGAGTCCGT-3′
Primer pRSF-SeAR-inf-F:
(SEQ ID NO: 7)
5′-aaggagatatacatatgATGTTCGGTCTTATCGGTCA-3′
Primer pRSF-SeAR-inf-R:
(SEQ ID NO: 8)
5′-ttgagatctgccatatgTCAAATTGCCAATGCCAAGG-3′

Subsequently, PstI-treated pRSF-1b (Novagen) was ligated to the NpADvo fragment using the In-Fusion HD Cloning kit (Invitrogen), the resulting plasmid was further digested with NdeI, and the resultant was bound to the SeAAvo fragment using the aforementioned kit. The vector thus obtained was designated as pRSF-NpAD-SeAR. PCR was then carried out under the conditions described below using the resulting pRSF-NpAD-SeAR as a template.

TABLE 4
Reaction composition:

| | | |
|---|---|---|
| pRSF-NpAD-SeAR (1 ng/μl) | 1 | μl |
| 10x Pfu Ultra II reaction buffer | 5 | μl |
| dNTP mix (25 mM each dNTP) | 0.5 | μl |
| Primer Fwl (10 μM) | 0.5 | μl |
| Primer Rvl (10 μM) | 0.5 | μl |
| Pfu Ultra II fusion HS DNA polymerase | 1 | μl |
| Sterilized water | 41.5 | μl |
| Total | 50 | μl |

PCR temperature conditions comprises: 95° C. for 2 minutes, a cycle of 95° C. for 20 seconds, 55° for 20 seconds, and 72° C. for 30 seconds repeated 25 times, and 72° C. for 3 minutes. Primer sequences are as shown below.

Primer FW1:
(SEQ ID NO: 9)
AGGAGATATACCATGCAGCAGCTTACAGACC
Primer Rv1:
(SEQ ID NO: 10)
GCTCGAATTCGGATCTTACACCACATCATCTTCGGCACCTGGCATGG
CAACGCCAGCACCTATGAGTCCGTAGG

Subsequently, the PCR-amplified DNA fragment was inserted into a region between the NcoI site and the BamHI site of pRSF-Duet-1 using the In-Fusion HD Cloning kit (CloneTech). The resulting plasmid was designated as pRSF-NpAD-PA.

2.3.2: Preparation of pCDF-SeAR

Also, PCR was carried out under the conditions described below using pRSF-NpAD-SeAR as a template.

TABLE 5
Reaction composition:

| | | |
|---|---|---|
| pRSF-NpAD-SeAR (1 ng/μl) | 1 | μl |
| 10x Pfu Ultra II reaction buffer | 5 | μl |
| dNTP mix (25 mM each dNTP) | 1 | μl |
| Primer Fw2 (10 μM) | 2 | μl |
| Primer Rv2 (10 μM) | 2 | μl |
| Pfu Ultra II fusion HS DNA polymerase | 1 | μl |
| Sterilized water | 38 | μl |
| Total | 50 | μl |

PCR temperature conditions comprises: 92° C. for 2 minutes, a cycle of 92° C. for 10 seconds, 55° for 20 seconds, and 68° C. for 5 minutes repeated 25 times, and 72° C. for 3 minutes. Primer sequences are as shown below.

Primer FW2:
(SEQ ID NO: 11)
AAGGAGATATACATATGATGTTCGGTCTTATCGGTCA
Primer Rv2:
(SEQ ID NO: 12)
TTGAGATCTGCCATATGTCAAATTGCCAATGCCAAGG

Subsequently, the PCR-amplified DNA fragment was inserted into the NdeI site of pCDD-Duet-1 using the In-Fusion HD Cloning kit (CloneTech). The resulting plasmid was designated as pCDF-SeAR.

2.3.3: Preparation of Plasmid for NpAD Mutant Gene Expression

Subsequently, PCR was carried out under the conditions described below with the use of the pRSF-NpAD-PA obtained above as a template and a set of primers capable of introducing a substitution mutation into a given site. The sets of primers used in this example are summarized in Table 7.

TABLE 6
Reaction composition:

| | | |
|---|---|---|
| pRSF-NpAD-PA (10 ng/μl) | 0.5 | μl |
| 2x PrimeStar Max Premix | 12.5 | μl |
| Fw shown in Table 7 (10 μM) | 0.5 | μl |
| Rv shown in Table 7 (10 μM) | 0.5 | μl |
| PrimeStar DNA polymerase | 1 | μl |
| Sterilized water | 10 | μl |
| Total | 25 | μl |

PCR temperature conditions comprises: a cycle of 98° C. for 10 seconds, 58° for 15 seconds, and 72° C. for 30 seconds repeated 30 times.

TABLE 7

| | | | | | SEQ |
|---|---|---|---|---|---|
| | Mutation | | | | ID |
| Plasmid | site | Primer | | Primer sequence | NO: |
| No. 1 | Y18F | Fw | NpAD_Y18F-F | GAAACATTTAAAGATGCTTATAGCCGGATTAATGC | 13 |
| | | Rv | NpAD_Y18F-R | ATCTTTAAATGTTTCGCTCTTGAAATCTAATTCTTTAG | 14 |
| No. 2 | V29T | Fw | NpAD_V29T-F | GCGATCACCATTGAAGGGGAACAAGAAGCCCA | 15 |
| | | Rv | NpAD_V29T-R | TTCAATGGTGATCGCATTAATCCGGCTATAAGC | 16 |
| No. 3 | V29M | Fw | NpAD_V29M-F | GCGATCATGATTGAAGGGGAACAAGAAGCCCA | 17 |
| | | Rv | NpAD_V29M-R | TTCAATCATGATCGCATTAATCCGGCTATAAGC | 18 |
| No. 4 | E35Y | Fw | NpAD_E35Y-F | GAACAATATGCCCATGAAAATTACATCACACTAG | 19 |
| | | Rv | NpAD_E35Y-R | ATGGGCATATTGTTCCCCTTCAATCACGATC | 20 |
| No. 5 | N39M | Fw | NpAD_N39M-F | CATGAAATGTACATCACACTAGCCCAACTGC | 21 |
| | | Rv | NpAD_N39M-R | GATGTACATTTCATGGGCTTCTTGTTCCCCTTC | 22 |
| No. 6 | N39T | Fw | NpAD_N39T-F | CATGAAACCTACATCACACTAGCCCAACTGC | 23 |
| | | Rv | NpAD_N39T-R | GATGTAGGTTTCATGGGCTTCTTGTTCCCCTTC | 24 |
| No. 7 | N39V | Fw | NpAD_N39V-F | CATGAAGTGTACATCACACTAGCCCAACTGC | 25 |
| | | Rv | NpAD_N39V-R | GATGTACACTTCATGGGCTTCTTGTTCCCCTTC | 26 |
| No. 8 | T42D | Fw | NpAD_T42D-F | TACATCGATCTAGCCCAACTGCTGCCAGAATC | 27 |
| | | Rv | NpAD_T42D-R | GGCTAGATCGATGTAATTTTCATGGGCTTCTTGTTC | 28 |
| No. 9 | T42N | Fw | NpAD_T42N-F | TACATCAACCTAGCCCAACTGCTGCCAGAATC | 29 |
| | | Rv | NpAD_T42N-R | GGCTAGGTTGATGTAATTTTCATGGGCTTCTTGTTC | 30 |
| No. 10 | A44S | Fw | NpAD_A44S-F | ACACTAAGCCAACTGCTGCCAGAATCTCATG | 31 |
| | | Rv | NpAD_A44S-R | CAGTTGGCTTAGTGTGATGTAATTTTCATGGGCTTC | 32 |
| No. 11 | H51Y | Fw | NpAD_H51Y-F | GAATCTTATGATGAATTGATTCGCCTATCCAAG | 33 |
| | | Rv | NpAD_H51Y-R | TTCATCATAAGATTCTGGCAGCAGTTGGGCTA | 34 |
| No. 12 | L54Q | Fw | NpAD_L54Q-F | GATGAACAGATTCGCCTATCCAAGATGGAAAGC | 35 |
| | | Rv | NpAD_L54Q-R | GCGAATCTGTTCATCATGAGATTCTGGCAGCAG | 36 |
| No. 13 | L54E | Fw | NpAD_L54E-F | GATGAAGAAATTCGCCTATCCAAGATGGAAAGC | 37 |
| | | Rv | NpAD_L54E-R | GCGAATTTCTTCATCATGAGATTCTGGCAGCAG | 38 |
| No. 14 | L54K | Fw | NpAD_L54K-F | GATGAAAAAATTCGCCTATCCAAGATGGAAAGC | 39 |
| | | Rv | NpAD_L54K-R | GCGAATTTTTTCATCATGAGATTCTGGCAGCAG | 40 |
| No. 15 | I55W | Fw | NpAD_I55W-F | GAATTGTGGCGCCTATCCAAGATGGAAAGCC | 41 |
| | | Rv | NpAD_I55W-R | TAGGCGCCACAATTCATCATGAGATTCTGGCAG | 42 |
| No. 16 | L57Q | Fw | NpAD_L57Q-F | ATTCGCCAGTCCAAGATGGAAAGCCGCCATAAG | 43 |
| | | Rv | NpAD_L57Q-R | CTTGGACTGGCGAATCAATTCATCATGAGATTCTC | 44 |
| No. 17 | L57E | Fw | NpAD_L57E-F | ATTCGCGAATCCAAGATGGAAAGCCGCCATAAG | 45 |
| | | Rv | NpAD_L57E-R | CTTGGATTCGCGAATCAATTCATCATGAGATTCTC | 46 |
| No. 18 | L57K | Fw | NpAD_L57K-F | ATTCGCAAATCCAAGATGGAAAGCCGCCATAAG | 47 |
| | | Rv | NpAD_L57K-R | CTTGGATTTGCGAATCAATTCATCATGAGATTCTC | 48 |
| No. 19 | L57W | Fw | NpAD_L57W-F | ATTCGCTGGTCCAAGATGGAAAGCCGCCATAAG | 49 |
| | | Rv | NpAD_L57W-R | CTTGGACCAGCGAATCAATTCATCATGAGATTCTC | 50 |
| No. 20 | L57Y | Fw | NpAD_L57Y-F | ATTCGCTATTCCAAGATGGAAAGCCGCCATAAG | 51 |
| | | Rv | NpAD_L57Y-R | CTTGGAATAGCGAATCAATTCATCATGAGATTCTC | 52 |
| No. 21 | S58N | Fw | NpAD_S58N-F | CGCCTAAACAAGATGGAAAGCCGCCATAAG | 53 |
| | | Rv | NpAD_S58N-R | CATCTTGTTTAGGCGAATCAATTCATCATGAG | 54 |
| No. 22 | M60D | Fw | NpAD_M60D-F | TCCAAGGATGAAAGCCGCCATAAGAAAGGATTTG | 55 |
| | | Rv | NpAD_M60D-R | GCTTTCATCCTTGGATAGGCGAATCAATTCATC | 56 |
| No. 23 | M60N | Fw | NpAD_M60N-F | TCCAAGAACGAAAGCCGCCATAAGAAAGGATTTG | 57 |
| | | Rv | NpAD_M60N-R | GCTTTCGTTCTTGGATAGGCGAATCAATTCATC | 58 |
| No. 24 | E61T | Fw | NpAD_E61T-F | AAGATGACCAGCCGCCATAAGAAAGGATTTG | 59 |
| | | Rv | NpAD_E61T-R | GCGGCTGGTCATCTTGGATAGGCGAATCAATTC | 60 |
| No. 25 | K65Y | Fw | NpAD_K65Y-F | CGCCATTATAAAGGATTTGAAGCTTGTGGGCG | 61 |
| | | Rv | NpAD_K65Y-R | TCCTTTATAATGGCGGCTTTCCATCTTGGATAG | 62 |
| No. 26 | G67H | Fw | NpAD_G67H-F | AAGAAACATTTTGAAGCTTGTGGGCGCAATTTAG | 63 |
| | | Rv | NpAD_G67H-R | TTCAAAATGTTTCTTATGGCGGCTTTCCATC | 64 |
| No. 27 | A70S | Fw | NpAD_A70S-F | TTTGAAAGCTGTGGGCGCAATTTAGCTGTTAC | 65 |
| | | Rv | NpAD_A70S-R | CCCACAGCTTTCAAATCCTTTCTTATGGCGGC | 66 |
| No. 28 | C71G | Fw | NpAD_C71G-F | GAAGCTGGCGGGCGCAATTTAGCTGTTACC | 67 |
| | | Rv | NpAD_C71G-R | GCGCCCGCCAGCTTCAAATCCTTTCTTATGGC | 68 |
| No. 29 | S89N | Fw | NpAD_S89N-F | TTTTTCAACGGCCTACACCAAAATTTTCAAACAG | 69 |
| | | Rv | NpAD_S89N-R | TAGGCCGTTGAAAAACTCTTTGGCAAATTGCAAATC | 70 |
| No. 30 | N94V | Fw | NpAD_N94V-F | CACCAAGTGTTTCAAACAGCTGCCGCAGAAG | 71 |
| | | Rv | NpAD_N94V-R | TTGAAACACTTGGTGTAGGCCGGAGAAAAACTC | 72 |
| No. 31 | A100S | Fw | NpAD_A100S-F | GCTGCCAGCGAAGGGAAAGTGGTTACTTGTC | 73 |
| | | Rv | NpAD_A100S-R | CCCTTCGCTGGCAGCTGTTTGAAAATTTTGGTG | 74 |
| No. 32 | V104A | Fw | NpAD_V104A-F | GGGAAAGCGGTTACTTGTCTGTTGATTCAGTC | 75 |
| | | Rv | NpAD_V104A-R | AGTAACCGCTTTCCCTTCTGCGGCAGCTGTTTG | 76 |
| No. 33 | V104N | Fw | NpAD_V104N-F | GGGAAAAACGTTACTTGTCTGTTGATTCAGTC | 77 |
| | | Rv | NpAD_V104N-R | AGTAACGTTTTTCCCTTCTGCGGCAGCTGTTTG | 78 |
| No. 34 | T106V | Fw | NpAD_T106V-F | GTGGTTGTGTGCTGTTGATTCAGTCTTTAATTATTG | 79 |
| | | Rv | NpAD_T106V-R | CAGACACACAACCACTTTCCCTTCTGCGG | 80 |
| No. 35 | T106M | Fw | NpAD_T106M-F | GTGGTTATGTGTCTGTTGATTCAGTCTTTAATTATTG | 81 |
| | | Rv | NpAD_T106M-R | CAGACACATAACCACTTTCCCTTCTGCGG | 82 |
| No. 36 | Q111L | Fw | NpAD_Q111L-F | TTGATTCTGTCTTTAATTATTGAATGTTTTGCGATC | 83 |
| | | Rv | NpAD_Q111L-R | TAAAGACAGAATCAACAGACAAGTAACCACTTTCC | 84 |
| No. 37 | Q111I | Fw | NpAD_Q111I-F | TTGATTATTTCTTTAATTATTGAATGTTTTGCGATC | 85 |
| | | Rv | NpAD_Q111I-R | TAAAGAAATAATCAACAGACAAGTAACCACTTTCC | 86 |
| No. 38 | Y123H | Fw | NpAD_Y123H-F | GCAGCACATAACATTTACATCCCCGTTGCCGACG | 87 |
| | | Rv | NpAD_Y123H-R | AATGTTATGTGCTGCGATCGCAAAACATTC | 88 |
| No. 39 | Y126H | Fw | NpAD_Y126H-F | AACATTCATATCCCCGTTGCCGACGATTTCG | 89 |
| | | Rv | NpAD_Y126H-R | GGGGATATGAATGTTATATGCTGCGATCGCAAAAC | 90 |
| No. 40 | E144D | Fw | NpAD_E144D-F | GTTAAAGATGAATACAGCCACCTCAATTTTG | 91 |
| | | Rv | NpAD_E144D-R | GTATTCATCTTTAACTACTCCTTCAGTAATTTTAC | 92 |
| No. 41 | E144V | Fw | NpAD_E144V-F | GTTAAAGTGGAATACAGCCACCTCAATTTTG | 93 |
| | | Rv | NpAD_E144V-R | GTATTCCACTTTAACTACTCCTTCAGTAATTTTAC | 94 |
| No. 42 | V154E | Fw | NpAD_V154E-F | GGAGAAGAATGGTTGAAAGAACACTTTGCAG | 95 |
| | | Rv | NpAD_V154E-R | CAACCATTCTTCTCCAAAATTGAGGTGGCTG | 96 |
| No. 43 | A161S | Fw | NpAD_A161S-F | CACTTTAGCGAATCCAAAGCTGAACTTGAAC | 97 |
| | | Rv | NpAD_A161S-R | GGATTCGCTAAAGTGTTCTTTCAACCAAACTTC | 98 |
| No. 44 | L169T | Fw | NpAD_L169T-F | CTTGAAACCGCAAATCGCCAGAACCTACCCATC | 99 |
| | | Rv | NpAD_L169T-R | ATTTGCGGTTTCAAGTTCAGCTTTGGATTCTGCAAAGTG | 100 |
| No. 45 | L169A | Fw | NpAD_L169A-F | CTTGAAGCGGCAAATCGCCAGAACCTACCCATC | 101 |
| | | Rv | NpAD_L169A-R | ATTTGCCGCTTCAAGTTCAGCTTTGGATTCTG | 102 |
| No. 46 | L169Y | Fw | NpAD_L169Y-F | CTTGAATATGCAAATCGCCAGAACCTACCCATC | 103 |
| | | Rv | NpAD_L169Y-R | ATTTGCATATTCAAGTTCAGCTTTGGATTCTGCAAAGTG | 104 |
| No. 47 | I155Y | Fw | NpAD_I155Y-F | GAATTGTATCGCCTATCCAAGATGGAAAGCC | 105 |
| | | Rv | NpAD_Y I155Y-R | TAGGCGATACAATTCATCATGAGATTCTGGCAG | 106 |
| No. 48 | N124V | Fw | NpAD_N124V-F_2 | GCATATGTGATTTACATCCCCGTTGCCGAC | 107 |
| | | Rv | NpAD_N124V-R_2 | GTAAATCACATATGCTGCGATCGCAAAACATTC | 108 |
| No. 49 | L169W | Fw | NpAD_L196W-F | CTTGAATGGGCAAATCGCCAGAACCTACCCATC | 109 |
| | | Rv | NpAD_L196W-R | ATTTGCCCATTCAAGTTCAGCTTTGGATTCTGCAAAG | 110 |
| No. 50 | N174M | Fw | NpAD_N174M-F | CGCCAGATGCTACCCATCGTCTGGAAAATG | 111 |
| | | Rv | NpAD_N174M-R | GGGTAGCATCTGGCGATTTGCAAGTTCAAGTTC | 112 |
| No. 51 | N174T | Fw | NpAD_N174T-F | CGCCAGACCCTACCCATCGTCTGGAAAATGC | 113 |
| | | Rv | NpAD_N174T-R | GGGTAGGGTCTGGCGATTTGCAAGTTCAAG | 114 |
| No. 52 | L175Q | Fw | NpAD_L175Q-F | CAGAACCAGCCCATCGTCTGGAAAATGCTCAAC | 115 |
| | | Rv | NpAD_L175Q-R | GATGGGCTGGTTCTGGCGATTTGCAAGTTCAAG | 116 |
| No. 53 | L175E | Fw | NpAD_L175E-F | CAGAACGAACCCATCGTCTGGAAAATGCTCAAC | 117 |
| | | Rv | NpAD_L175E-R | GATGGGTTCGTTCTGGCGATTTGCAAGTTCAAG | 118 |
| No. 54 | L175K | Fw | NpAD_L175K-F | CAGAACAAACCCATCGTCTGGAAAATGCTCAACCAAG | 119 |
| | | Rv | NpAD_L175K-R | GATGGGTTTGTTCTGGCGATTTGCAAGTTCAAG | 120 |
| No. 55 | I177Y | Fw | NpAD_I177Y-F | CTACCCTATGTCTGGAAAATGCTCAACCAAGTAG | 121 |
| | | Rv | NpAD_I177Y-R | CCAGACATAGGGTAGGTTCTGGCGATTTGCAAG | 122 |
| No. 56 | I177W | Fw | NpAD_I177W-F | CTACCCTGGGTCTGGAAAATGCTCAACCAAGTAG | 123 |
| | | Rv | NpAD_I177YW-R | CCAGACCCAGGGTAGGTTCTGGCGATTTGCAAGTTCAAG | 124 |
| No. 57 | D188V | Fw | NpAD_D188V-F | GAAGGTGTGGCCCACACAATGGCAATGGAAAA | 125 |
| | | Rv | NpAD_D188V-R | GTGGGCCACACCTTCTACTTGGTTGAGCATTTTCC | 126 |
| No. 58 | T191V | Fw | NpAD_T191V-F | GCCCACGTGATGGCAATGGAAAAAGATGCTTTGG | 127 |
| | | Rv | NpAD_T191V-F | TGCCATCACGTGGGCATCACCTTCTACTTGGTTGAGC | 128 |
| No. 59 | T191I | Fw | NpAD_T191I-F | GCCCACATTATGGCAATGGAAAAAGATGCTTTGG | 129 |
| | | Rv | NpAD_T191I-R | TGCCATAATGTGGGCATCACCTTCTACTTGG | 130 |
| No. 60 | M192L | Fw | NpAD_M192L-F | CACACACTGGCAATGGAAAAAGATGCTTTGGT | 131 |
| | | Rv | NpAD_M192L-R | CATTGCCAGTGTGTGGGCATCACCTTCTACTTG | 132 |
| No. 61 | K196L | Fw | NpAD_K196L-F | ATGGAACTGGATGCTTTGGTAGAAGACTTCATGATTC | 133 |
| | | Rv | NpAD_K196L-R | AGCATCCAGTTCCATTGCCATTGTGTGGGC | 134 |
| No. 62 | K196I | Fw | NpAD_K196I-F | ATGGAAATTGATGCTTTGGTAGAAGACTTCATGATTC | 135 |
| | | Rv | NpAD_K196I-R | AGCATCAATTTCCATTGCCATTGTGTGGGCATC | 136 |
| No. 63 | D197E | Fw | NpAD_D197E-F | GAAAAAGAAGCTTTGGTAGAAGACTTCATG | 137 |
| | | Rv | NpAD_D197E-R | CAAAGCTTCTTTTTCCATTGCCATTGTGTGGGCATC | 138 |
| No. 64 | D197Y | Fw | NpAD_D197Y-F | GAAAAATATGCTTTGGTAGAAGACTTCATGAT | 139 |
| | | Rv | NpAD_D197Y-R | CAAAGCATATTTTTCCATTGCCATTGTGTGG | 140 |
| No. 65 | H201Y | Fw | NpAD_H201Y-F | TTGGTATATGACTTCATGATTCAGTATGGTG | 141 |
| | | Rv | NpAD_H201Y-R | GAAGTCATATACCAAAGCATCTTTTTCCATTG | 142 |
| No. 66 | G208H | Fw | NpAD_G208H-F | CAGTATCATGAAGCATTGAGTAACATTGG | 143 |
| | | Rv | NpAD_G208H-R | TGCTTCATGATACTGAATCATGAAGTCTTCTACC | 144 |

The 4.5-kb DNA fragment amplified via PCR was purified. With the use of the purified DNA fragment, the E. coli JM109 strain was transformed. The nucleotide sequences of the mutant decarbonylase genes included in the plasmids (No. 1 to No. 66) obtained from the transformant were determined to confirm the introduction of the mutation of interest and the absence of mutations in other regions.

2.4: Evaluation of Mutant Decarbonylase Gene

The E. coli BL-21 strain was transformed with the use of the plasmids No. 1 to No. 66 and pCDF-SeAR obtained above to prepare transformants. pRSF-NpAD-PA was used instead of the plasmids No. 1 to No. 66, and the transformants prepared with the use of pRSF-NpAD-PA and pCDF-SeAR were designated as wild-type strains. The wild-type strains and the transformants were cultured and the amounts of hydrocarbon production were quantitatively compared via MG/CMS.

In this example, the amount of hydrocarbon produced by the wild-type strain at O.D. 600 nm was designated to be 1, and the hydrocarbon-producing capacity of a transformant in which the mutant decarbonylase gene had been expressed was evaluated relative thereto.

Culture was conducted by first inoculating transformants into a 14-ml round tube (BD Falcon) containing 3 ml of the LB Broth Miller medium (Luria-Bertani, Difco) containing necessary antibiotics and performing agitation culture at 100 strokes/min for 18 hours at 37° C. using a three-tier culture vessel (MW-312, ABLE). The resulting preculture solution was inoculated at a concentration of 1% in 3 ml of an M9YE medium containing antibiotics, and culture was conducted with the use of a disposable glass test tube (φ16 mm×150 mm, manufactured by IWAKI) and the same culture vessel at 30° C. and 90 strokes/min for 2 or 3 days. In this culture, IPTG was added to a final concentration of 1 mM 4 hours after the transformants were inoculated.

Ethyl acetate (3 ml) was added to the equivalent amount of the culture solution 2 or 3 days after the initiation of culture and the resultant was blended using a vortex mixer for 10 seconds. After the mixture was centrifuged using a centrifuge (LC-230, TOMY) at room temperature and 2,000 rpm for 10 minutes, 1 ml of the ethyl acetate layer was transferred to a GC/MS vial, 10 ml of the internal standard solution (1 μl/ml R-(−)-2-octanol/ethanol) was added thereto, and the vial was fastened.

A method of quantification via GC/MS is as described below. At the outset, recombinants grown on the agarose plate were inoculated into the 14-ml round tube (BD Falcon) containing 3 ml of the aforementioned medium, and culture was then conducted using a three-tier culture vessel (MW-312, ABLE) at 130 strokes/min for 18 hours at a given temperature. The resulting preculture solution was inoculated at a concentration of 1% in 3 ml of an M9YE medium containing antibiotics in a disposable glass test tube (φ16×150 mm, IWAKI), culture was conducted in the same manner at 90 strokes/min for 4 hours, IPTG (final concentration: 1 mM) was added thereto, and culture was then conducted for an additional 3 days.

After the culture, 1.5 ml of the culture solution was fractionated in an Eppendorf tube and centrifuged using a small centrifuge (MX-301, TOMY) at 24° C. and 5800 g for 1 minute. The supernatant was removed while retaining 50 μl thereof, and strains were suspended. Subsequently, 150 μl of ethyl acetate was added, the resultant was vigorously blended using a vortex mixer for multiple samples (Mixer 5432, Eppendorf) for 5 minutes, the resultant was centrifuged in the same manner at 24° C. and 13000 g for 1 minute, and 100 μl of the ethyl acetate layer was transferred to the GC/MS vial. Thereafter, 50 μl of the internal standard solution (0.4% (v/v) 2-octanol dissolved in 2-propanol) was added and the resultant was subjected to GC/MS (7890GC/5975MSD, Agilent). Analytical conditions are described below.

TABLE 8
<GC/MS analysis conditions>

Detector: MS
MS zone temperature
MS Quad: 150° C.
MS Source: 230° C.
Interface temperature: 260° C.
Column: HP-5MS, Agilent (0.25 mm Φ × 30 m; film thickness:
0.25 μm)
Column temperature: retention at 60° C. for 1 min; temperature
increase at 50° C./min;
retention at 300° C. for 1 min
Inlet temperature: 250° C.
Amount of injection: 1 μl
Split ratio: 20:1
Carrier gas: He
Carrier gas flow rate: 1 ml/min
MS scan parameters
Low mass: 45
High mass: 350
Threshold: 30

[3. Results]

While a detailed description is omitted, the decarbonylase derived from the Nostoc punctiforme PCC73102 strain used in this example was subjected to modeling analysis. As a result, the decarbonylase of interest was found to be a protein with a special structure consisting of 10 α helices (Helix 1 to Helix 10 sequentially from the N terminus). Meanwhile, it is known that α helices are unstabilized and denatured because of the imbalance between hydrophobicity and hydrophilicity of the α helices (WO 2016/199898). In order to modify the imbalance, accordingly, a mutation causing amino acid substitution was introduced into a decarbonylase, and the influence of such mutation imposed on the amount of hydrocarbon production was investigated.

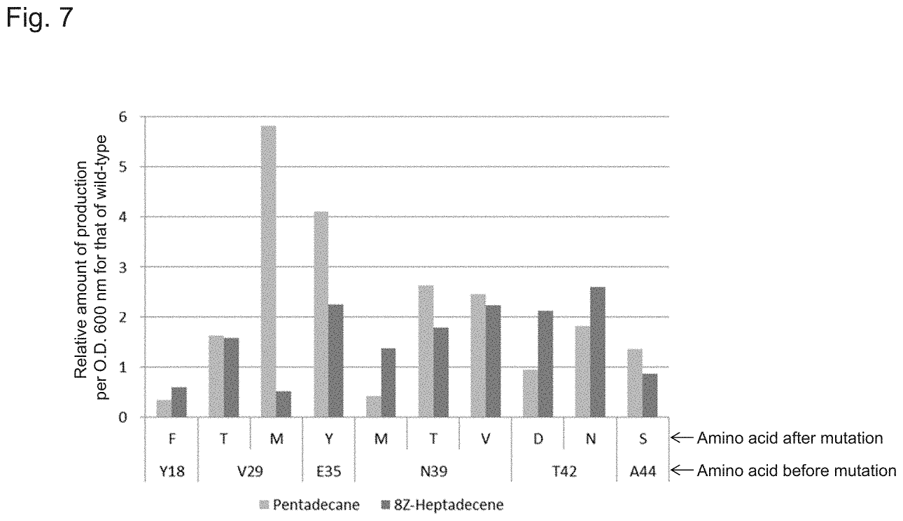

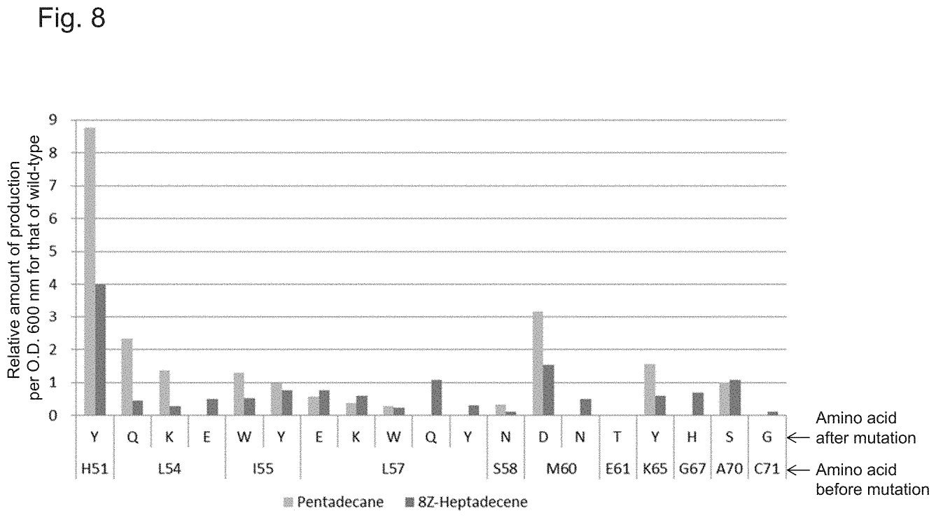

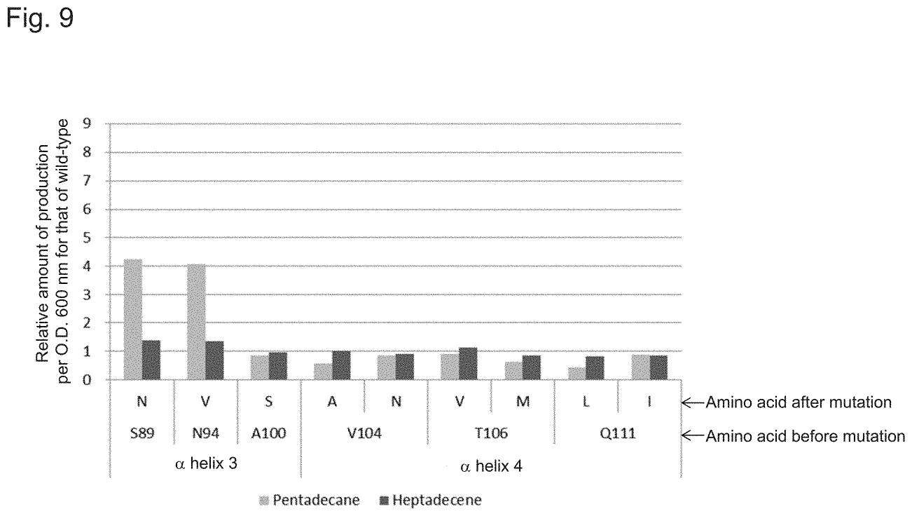

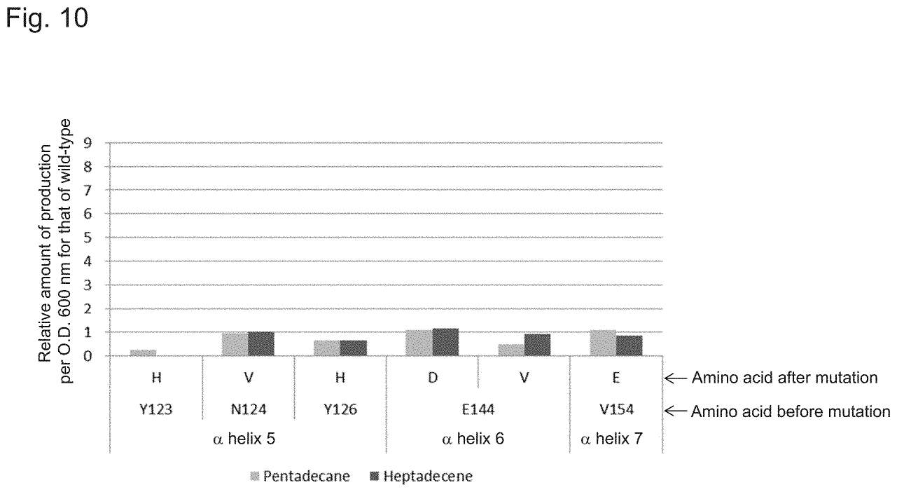

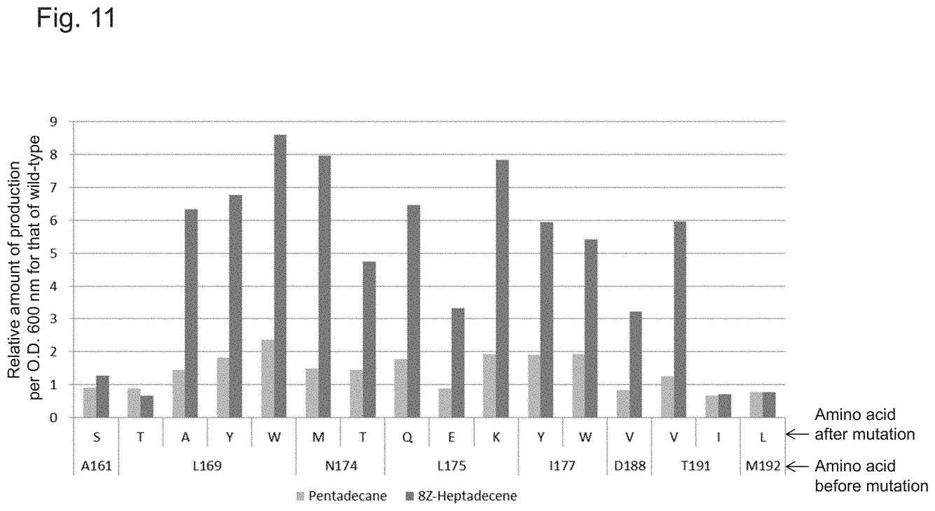

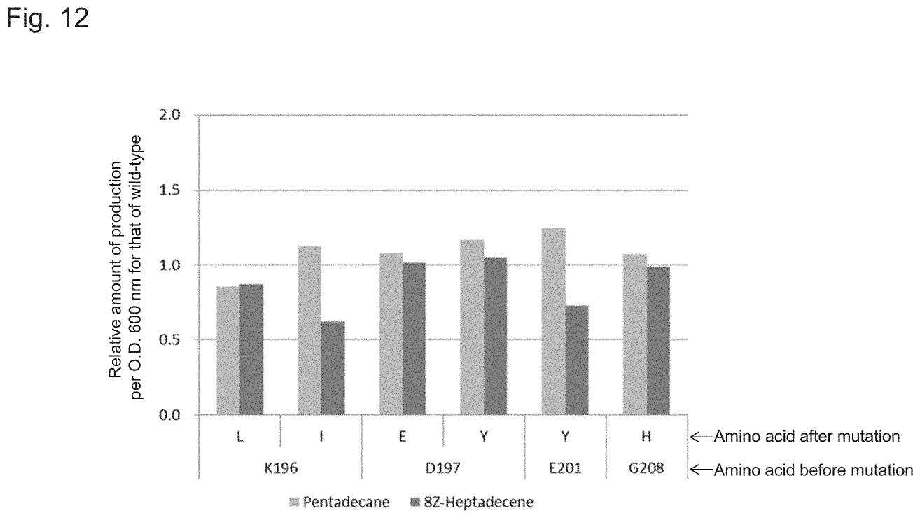

FIG. 7 shows the results of measuring the amount of hydrocarbons (pentadecane and heptadecane) produced by a transformant comprising a substitution mutation introduced into Helix 1. FIG. 8 shows the results of measuring the amount of hydrocarbons produced by a transformant comprising a substitution mutation introduced into Helix 2. FIG. 9 shows the results of measuring the amount of hydrocarbons produced by a transformant comprising mutations causing substitutions introduced into Helix 3 and Helix 4. FIG. 10 shows the results of measuring the amount of hydrocarbons produced by a transformant comprising mutations causing substitutions introduced into Helices 5, 6, and 7. FIG. 11 shows the results of measuring the amount of hydrocarbons produced by a transformant comprising a substitution mutation introduced into Helix 8. FIG. 12 shows the results of measuring the amount of hydrocarbons produced by a transformant comprising a substitution mutation introduced into Helix 9.

As shown in FIG. 7, hydrocarbon productivity is improved to a significant extent when valine at position 29, glutamic acid at position 35, asparagine at position 39, and threonine at position 42 in Helix 1 are substituted to resolve α helix instability. As shown in FIG. 8, hydrocarbon productivity is improved to a significant extent when histidine at position 51, leucine at position 54, and methionine at position 60 in Helix 2 are substituted to resolve α helix instability. As shown in FIG. 9, hydrocarbon productivity is improved to a significant extent when serine at position 89 and asparagine at position 94 in Helix 3 are substituted to resolve α helix instability. As shown in FIG. 11, in addition, hydrocarbon productivity is improved to a significant extent when leucine at position 169, asparagine at position 174, leucine at position 175, isoleucine at position 177, and aspartic acid at position 188 in Helix 8 are substituted to resolve α helix instability.

On the contrary, mutations causing substitutions in Helices 4 to 7 and 9 were found to impose no influence on hydrocarbon productivity (FIGS. 9, 10, and 12).

In particular, mutations causing substitutions that had enhanced pentadecane productivity by 3 times or more compared with wild-type strains were H51Y (8.77 times), V29M (5.82 times), S89N (4.25 times), E35Y (4.11 times), N94V (4.08 times), and M60D (3.16 times). Mutations causing substitutions that had enhanced heptadecane productivity by 3 times or more compared with wild-type strains were L169W (8.59 times), N174M (7.95 times), L175K (7.82 times), L169Y (6.77 times), L175Q (6.45 times), L169A (6.32 times), T191V (5.95 times), I177Y (5.93 times), I177W (5.42 times), N174T (4.75 times), H51Y (3.98 times), L175E (3.32 times), and D188V (3.21 times).

What is claimed is:

1. A mutant decarbonylase gene encoding a decarbonylase mutant having at least one substitution mutation, wherein the mutation is selected from the group consisting of:
a substitution mutation of an amino acid corresponding to valine at position 29 in the amino acid sequence as shown in SEQ ID NO: 2 with an amino acid with a low degree of hydrophobicity;
a substitution mutation of an amino acid corresponding to glutamic acid at position 35 in the amino acid sequence as shown in SEQ ID NO: 2 with an amino acid with a high degree of hydrophobicity;
a substitution mutation of an amino acid corresponding to asparagine at position 39 in the amino acid sequence as shown in SEQ ID NO: 2 with an amino acid with a high degree of hydrophobicity;
a substitution mutation of an amino acid corresponding to threonine at position 42 in the amino acid sequence as shown in SEQ ID NO: 2 with an amino acid with a low degree of hydrophobicity;
a substitution mutation of an amino acid corresponding to histidine at position 51 in the amino acid sequence as shown in SEQ ID NO: 2 with an amino acid with a high degree of hydrophobicity;
a substitution mutation of an amino acid corresponding to leucine at position 54 in the amino acid sequence as shown in SEQ ID NO: 2 with an amino acid with a low degree of hydrophobicity;
a substitution mutation of an amino acid corresponding to methionine at position 60 in the amino acid sequence as shown in SEQ ID NO: 2 with an amino acid with a low degree of hydrophobicity;
a substitution mutation of an amino acid corresponding to serine at position 89 in the amino acid sequence as shown in SEQ ID NO: 2 with an amino acid with a low degree of hydrophobicity;
a substitution mutation of an amino acid corresponding to asparagine at position 94 in the amino acid sequence as shown in SEQ ID NO: 2 with an amino acid with a high degree of hydrophobicity;
a substitution mutation of an amino acid corresponding to leucine at position 169 in the amino acid sequence as shown in SEQ ID NO: 2 with an amino acid with a low degree of hydrophobicity;
a substitution mutation of an amino acid corresponding to asparagine at position 174 in the amino acid sequence as shown in SEQ ID NO: 2 with an amino acid with a high degree of hydrophobicity;
a substitution mutation of an amino acid corresponding to leucine at position 175 in the amino acid sequence as shown in SEQ ID NO: 2 with an amino acid with a low degree of hydrophobicity;
a substitution mutation of an amino acid corresponding to isoleucine at position 177 in the amino acid sequence as shown in SEQ ID NO: 2 with an amino acid with a low degree of hydrophobicity; and
a substitution mutation of an amino acid corresponding to aspartic acid at position 188 in the amino acid sequence as shown in SEQ ID NO: 2 with an amino acid with a high degree of hydrophobicity.

2. The mutant decarbonylase gene according to claim 1, wherein the substitution mutation of an amino acid corresponding to valine at position 29 is a substitution mutation with an amino acid selected from the group consisting of tyrosine, tryptophan, serine, glycine, alanine, methionine, cysteine, phenylalanine, and leucine.

3. The mutant decarbonylase gene according to claim 1, wherein the substitution mutation of an amino acid corresponding to valine at position 29 is a substitution mutation with an amino acid selected from the group consisting of tyrosine, tryptophan, serine, glycine, alanine, and methionine.

4. The mutant decarbonylase gene according to claim 1, wherein the substitution mutation of an amino acid corresponding to valine at position 29 is a substitution mutation with methionine.

5. The mutant decarbonylase gene according to claim 1, wherein the substitution mutation of an amino acid corresponding to glutamic acid at position 35 is a substitution mutation with an amino acid selected from the group consisting of proline, tyrosine, tryptophan, serine, threonine, glycine, alanine, methionine, cysteine, phenylalanine, leucine, valine, and isoleucine.

6. The mutant decarbonylase gene according to claim 1, wherein the substitution mutation of an amino acid corresponding to glutamic acid at position 35 is a substitution mutation with an amino acid selected from the group consisting of proline, tyrosine, tryptophan, serine, threonine, and glycine.

7. The mutant decarbonylase gene according to claim 1, wherein the substitution mutation of an amino acid corresponding to glutamic acid at position 35 is a substitution mutation with tyrosine.

8. The mutant decarbonylase gene according to claim 1, wherein the substitution mutation of an amino acid corresponding to asparagine at position 39 is a substitution mutation with an amino acid selected from the group consisting of glycine, alanine, cysteine, phenylalanine, leucine, valine, and isoleucine.

9. The mutant decarbonylase gene according to claim 1, wherein the substitution mutation of an amino acid corresponding to asparagine at position 39 is a substitution mutation with an amino acid selected from the group consisting of cysteine, phenylalanine, leucine, valine, and isoleucine.

10. The mutant decarbonylase gene according to claim 1, wherein the substitution mutation of an amino acid corresponding to asparagine at position 39 is a substitution mutation with valine.

11. The mutant decarbonylase gene according to claim 1, wherein the substitution mutation of an amino acid corresponding to threonine at position 42 is a substitution mutation with an amino acid selected from the group consisting of arginine, lysine, glutamine, asparagine, aspartic acid, glutamic acid, histidine, proline, tyrosine, tryptophan, serine, and glycine.

12. The mutant decarbonylase gene according to claim 1, wherein the substitution mutation of an amino acid corresponding to threonine at position 42 is a substitution mutation with an amino acid selected from the group consisting of lysine, glutamine, asparagine, aspartic acid, glutamic acid, histidine, proline, and tyrosine.

13. The mutant decarbonylase gene according to claim 1, wherein the substitution mutation of an amino acid corresponding to threonine at position 42 is a substitution mutation with asparagine or aspartic acid.

14. The mutant decarbonylase gene according to claim 1, wherein the substitution mutation of an amino acid corresponding to histidine at position 51 is a substitution mutation with an amino acid selected from the group consisting of proline, tyrosine, tryptophan, serine, threonine, and glycine.

15. The mutant decarbonylase gene according to claim 1, wherein the substitution mutation of an amino acid corresponding to histidine at position 51 is a substitution mutation with proline or tyrosine.

16. The mutant decarbonylase gene according to claim 1, wherein the substitution mutation of an amino acid corresponding to histidine at position 51 is a substitution mutation with tyrosine.

17. The mutant decarbonylase gene according to claim 1, wherein the substitution mutation of an amino acid corresponding to leucine at position 54 is a substitution mutation with an amino acid selected from the group consisting of glutamine, asparagine, aspartic acid, histidine, proline, tyrosine, tryptophan, serine, threonine, and glycine.

18. The mutant decarbonylase gene according to claim 1, wherein the substitution mutation of an amino acid corresponding to leucine at position 54 is a substitution mutation with an amino acid selected from the group consisting of glutamine, asparagine, aspartic acid, histidine, proline, and tyrosine.

19. The mutant decarbonylase gene according to claim 1, wherein the substitution mutation of an amino acid corresponding to leucine at position 54 is a substitution mutation with glutamine.

20. The mutant decarbonylase gene according to claim 1, wherein the substitution mutation of an amino acid corresponding to methionine at position 60 is a substitution mutation with an amino acid selected from the group consisting of glutamine, aspartic acid, glutamic acid, histidine, proline, and tyrosine.

21. The mutant decarbonylase gene according to claim 1, wherein the substitution mutation of an amino acid corresponding to methionine at position 60 is a substitution mutation with an amino acid selected from the group consisting of glutamine, aspartic acid, and glutamic acid.

22. The mutant decarbonylase gene according to claim 1, wherein the substitution mutation of an amino acid corresponding to methionine at position 60 is a substitution mutation with aspartic acid.

23. The mutant decarbonylase gene according to claim 1, wherein the substitution mutation of an amino acid corresponding to serine at position 89 is a substitution mutation with an amino acid selected from the group consisting of glutamine, asparagine, aspartic acid, glutamic acid, histidine, proline, and tyrosine.

24. The mutant decarbonylase gene according to claim 1, wherein the substitution mutation of an amino acid corresponding to serine at position 89 is a substitution mutation with an amino acid selected from the group consisting of glutamine, asparagine, aspartic acid, and glutamic acid.

25. The mutant decarbonylase gene according to claim 1, wherein the substitution mutation of an amino acid corresponding to serine at position 89 is a substitution mutation with asparagine.

26. The mutant decarbonylase gene according to claim 1, wherein the substitution mutation of an amino acid corresponding to asparagine at position 94 is a substitution mutation with an amino acid selected from the group consisting of cysteine, phenylalanine, leucine, valine, and isoleucine.

27. The mutant decarbonylase gene according to claim 1, wherein the substitution mutation of an amino acid corresponding to asparagine at position 94 is a substitution mutation with an amino acid selected from the group consisting of leucine, valine, and isoleucine.

28. The mutant decarbonylase gene according to claim 1, wherein the substitution mutation of an amino acid corresponding to asparagine at position 94 is a substitution mutation with valine.

29. The mutant decarbonylase gene according to claim 1, wherein the substitution mutation of an amino acid corresponding to leucine at position 169 is a substitution mutation with an amino acid selected from the group consisting of proline, tyrosine, tryptophan, serine, threonine, glycine, alanine, and methionine.

30. The mutant decarbonylase gene according to claim 1, wherein the substitution mutation of an amino acid corresponding to leucine at position 169 is a substitution mutation with an amino acid selected from the group consisting of tyrosine, tryptophan, serine, threonine, glycine, and alanine.

31. The mutant decarbonylase gene according to claim 1, wherein the substitution mutation of an amino acid corresponding to leucine at position 169 is a substitution mutation with an amino acid selected from the group consisting of tyrosine, tryptophan, and alanine.

32. The mutant decarbonylase gene according to claim 1, wherein the substitution mutation of an amino acid corresponding to asparagine at position 174 is a substitution mutation with an amino acid selected from the group consisting of proline, tyrosine, tryptophan, serine, threonine, glycine, alanine, methionine, cysteine, and phenylalanine.

33. The mutant decarbonylase gene according to claim 1, wherein the substitution mutation of an amino acid corresponding to asparagine at position 174 is a substitution mutation with an amino acid selected from the group consisting of tryptophan, serine, threonine, glycine, alanine, and methionine.

34. The mutant decarbonylase gene according to claim 1, wherein the substitution mutation of an amino acid corresponding to asparagine 174 is a substitution mutation with threonine or methionine.

35. The mutant decarbonylase gene according to claim 1, wherein the substitution mutation of an amino acid corresponding to leucine 175 is a substitution mutation with an amino acid selected from the group consisting of arginine, lysine, glutamine, asparagine, aspartic acid, glutamic acid, histidine, proline, and tyrosine.

36. The mutant decarbonylase gene according to claim 1, wherein the substitution mutation of an amino acid corresponding to leucine 175 is a substitution mutation with an amino acid selected from the group consisting of lysine, glutamine, asparagine, aspartic acid, glutamic acid, and histidine.

37. The mutant decarbonylase gene according to claim 1, wherein the substitution mutation of an amino acid corresponding to leucine 175 is a substitution mutation with an amino acid selected from the group consisting of lysine, glutamine, and glutamic acid.

38. The mutant decarbonylase gene according to claim 1, wherein the substitution mutation of an amino acid corresponding to isoleucine 177 is a substitution mutation with an amino acid selected from the group consisting of glutamine, asparagine, aspartic acid, glutamic acid, histidine, proline, tyrosine, tryptophan, serine, threonine, glycine, alanine, and methionine.

39. The mutant decarbonylase gene according to claim 1, wherein the substitution mutation of an amino acid corresponding to isoleucine 177 is a substitution mutation with an amino acid selected from the group consisting of proline, tyrosine, tryptophan, serine, threonine, and glycine.

40. The mutant decarbonylase gene according to claim 1, wherein the substitution mutation of an amino acid corresponding to isoleucine 177 is a substitution mutation with tyrosine or tryptophan.

41. The mutant decarbonylase gene according to claim 1, wherein the substitution mutation of an amino acid corresponding to aspartic acid 188 is a substitution mutation with an amino acid selected from the group consisting of cysteine, phenylalanine, leucine, valine, and isoleucine.

42. The mutant decarbonylase gene according to claim 1, wherein the substitution mutation of an amino acid corresponding to aspartic acid 188 is a substitution mutation with an amino acid selected from the group consisting of leucine, valine, and isoleucine.

43. The mutant decarbonylase gene according to claim 1, wherein the substitution mutation of an amino acid corresponding to aspartic acid 188 is a substitution mutation with valine.

44. The mutant decarbonylase gene according to claim 1, which has at least one substitution mutation selected from the group consisting of V29M, E35Y, N39T, N39V, T42D, T42N, H51Y, L54Q, M60D, S89N, N94V, L169A, L169Y, L169W, N174M, N174T, L175Q, L175E, L175K, I177Y, I177W, and D188V in the amino acid sequence as shown in SEQ ID NO: 2.

45. The mutant decarbonylase gene according to claim 1, which has H51Y and/or L169W in the amino acid sequence as shown in SEQ ID NO: 2.

46. A recombinant microorganism comprising the mutant decarbonylase gene according to claim 1 introduced into a host microorganism.

47. The recombinant microorganism according to claim 46, wherein the host microorganism is a bacterium of the genus Escherichia or Klebsiella.

48. A method for producing alkane comprising culturing the recombinant microorganism according to claim 46.

49. The method for producing alkane according to claim 48, which further comprises recovering alkane from a medium in which the recombinant microorganism is cultured.

50. The method for producing alkane according to claim 48, which further comprises recovering alkane from a medium in which the recombinant microorganism is cultured and purifying the recovered alkane.

51. The method for producing alkane according to claim 48, which further comprises producing alkane having 9 to 20 carbon atoms.
